(19) United States
(12) Patent Application Publication
(10) Pub. No.: US 2022/0023447 A1
(43) Pub. Date: Jan. 27, 2022

(54) FIBRIN-TARGETED POLYMERIZED SHELL LIPID MICROBUBBLES FOR DIAGNOSTIC AND THERAPEUTIC APPLICATIONS

(71) Applicants: NanoValent Pharmaceuticals Inc., Bozeman, MT (US); Trustees of Boston University, Boston, MA (US)

(72) Inventors: Jon Nagy, Bozeman, MT (US); Joyce Yun-Wei Wong, Chestnut Hill, MA (US); Benjamin J Keenan, Danvers, MA (US); Catherine Anne Gormley, Brookline, MA (US); Ray Glynn Holt, Framingham, MA (US)

(21) Appl. No.: 17/311,679

(22) PCT Filed: Dec. 6, 2019
(86) PCT No.: PCT/US2019/065092

§ 371 Date: Jun. 7, 2021

Related U.S. Application Data
(60) Provisional application No. 62/776,463, filed on Dec. 7, 2018.

(51) Int. Cl. A61K 49/22; A61K 47/69

(52) U.S. Cl. CPC A61K 49/223; A61K 47/6925; A61K 49/221

(57) ABSTRACT

Fibrin-targeted microbubbles and their use in ultrasound-based diagnostic and therapeutic applications are disclosed. In particular, the invention relates to the use of fibrin-targeted polymerized shell lipid microbubbles and methods of fabricating and using such fibrin-targeted microbubbles for ultrasound imaging of fibrin deposition in tissue, including adhesions and atherosclerotic plaques. The invention further relates to the use of such fibrin-targeted microbubbles as carriers for drug delivery and in ultrasound-based methods of treatment.

### RELATED APPLICATIONS

This application claims priority to U.S. Provisional Patent Application Ser. No. 62/776,463, filed on Dec. 7, 2018, which is hereby incorporated by reference herein in its entirety.

### FIELD OF THE INVENTION

The present invention pertains to the field of ultrasound and microbubble contrast agents. In particular, the invention relates to fibrin-targeted polymerized shell lipid microbubbles (PSMs) comprising polymerizable lipids, and methods of making and using such polymerized shell microbubbles in ultrasound-based diagnostic and therapeutic technologies.

### BACKGROUND OF THE INVENTION

Current ultrasound contrast agents (USCA) vary in composition from simple gas bubbles to albumin-coated bubbles to synthetic polymer bubbles. These vast differences in shell composition result in vastly differing properties, affecting key properties such as ultrasound response and in vivo circulation time. Likewise, size and size distribution exert effects over these properties. Clinically available ultrasound contrast agents are limited to polydisperse, non-targeted microbubbles, which result in non-specific highlighting of the vasculature. Targeted contrast ultrasound offers the potential to target specific molecular markers in the vasculature, peritoneal space, pericardium, and other non-vascular introductions, revealing information about molecular makeup in addition to structure. Information about molecular makeup is crucial in many diagnostic applications, such as inflammation in atherosclerosis and angiogenesis in cancer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to monodisperse and polydisperse polymerized shell lipid microbubbles (PSMs) comprising polymerizable lipids, and methods of making and using such PSMs in ultrasound-based diagnostic and therapeutic technologies. In particular, fibrin-targeted PSMs can be used in ultrasound imaging of adhesions, treatment of adhesions, and ultrasound-induced drug delivery.

In one aspect, the invention includes a fibrin-targeted microbubble comprising: a) a polymerized lipid shell; b) a fibrin-targeting agent, wherein the fibrin-targeting agent is conjugated to the polymerized lipid shell; and c) a gas core comprising at least one gas, wherein the gas core is encased within the polymerized lipid shell.

In certain embodiments, the polymerized lipid shell comprises at least one polymerizable lipid and at least one non-polymerizable lipid. In one embodiment, the polymerized lipid shell comprises at least about 5% polymerizable lipid. For example, the amount of the polymerizable lipid may range from about 5 to 50% of the total lipid in the polymerized lipid shell, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%.

In certain embodiments, at least one non-polymerizable or at least one polymerizable lipid is PEGylated. For example, in the fibrin-targeted microbubble, the amount of the PEGylated lipid may range from about 5% to about 50% of the total lipid in the polymerized lipid shell, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%.

In another embodiment, the non-polymerizable lipid is 1,2-distearoyl-sn-glycero-3-phosphocholine (DSPC), 1,2-distearoyl-sn-glycero-3-phosphoethanolamine-N-[methoxy(polyethylene glycol)-2000 (DSPE-PEG2000), or DSPE-PEG2000-biotin.

In another embodiment, the polymerizable lipid is a diacetylenic lipid.

In another embodiment, the fibrin-targeting agent is conjugated to the non-polymerizable lipid in the polymerized lipid shell.

In certain embodiments, the fibrin-targeting agent is a peptide, protein, antibody, antibody mimetic, or aptamer that selectively binds to fibrin.

In another embodiment, the fibrin-targeting agent is the CREKA fibrin-binding peptide or CPT-11. An exemplary fibrin-targeted microbubble with the CREKA fibrin-binding peptide comprises a polymerized lipid shell comprising 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide. In one embodiment, the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25.

Exemplary gases that may be used in the microbubbles include a perfluorocarbon (e.g., decafluorobutane), nitrogen, or air. In another embodiment, the gas core comprises at least two perfluorocarbons. In certain embodiments, the gas core of the microbubble comprises a heavy gas.

In another embodiment, the microbubble has an absorbance at a wavelength between about 400-580 nm. In another embodiment, the microbubble has an absorbance at a wavelength between about 400-650 nm

In another aspect, the invention includes a composition comprising a collection of microbubbles including a plurality of fibrin-targeted microbubbles as described herein. The microbubbles in the collection may be monodisperse or polydisperse. In one embodiment, the microbubbles in the collection range in size from about 3 μm to about 5 μm. In another embodiment, the microbubbles in the collection are monodisperse, wherein the monodispersity is about 10% of the average size of the microbubbles in the collection. In another embodiment, the average size of the microbubbles in the collection is between about 3 to about 5 μm.

In another embodiment, the composition further comprises a pharmaceutically acceptable excipient.

In certain embodiments, the composition comprises a collection of monodisperse microbubbles ranging in size from about 1.5 to about 2.5 μm in diameter, including any size within this range such as 1.5, 1.6, 1.7, 1.8, 1.9, 2.0, 2.1, 2.2, 2.3, 2.4, or 2.5 μm in diameter. In another embodiment, the monodisperse microbubbles have an average size of about 2 μm.

In other embodiments, the composition comprises a collection of polydisperse microbubbles ranging in size from about 0.5 to about 5 μm in diameter, including any size within this range such as 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, or 5.0 μm in diameter. In another embodiment, the polydisperse microbubbles have an average size of about 1.1 μm.

In another aspect, the invention includes a kit comprising a composition comprising fibrin-targeted microbubbles or reagents for producing fibrin targeted microbubbles, as described herein, and instructions for using the kit for ultrasound imaging for diagnosing or treating a fibrin-associated condition or disorder (e.g., adhesions or plaques). In one embodiment, the kit comprises fibrin-targeted microbubbles comprising a polymerized lipid shell comprising 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide. In another embodiment, the kit comprises 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide for preparing fibrin-targeted microbubbles. In another embodiment, the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25.

In another aspect, the invention includes a method of imaging tissue at a site of fibrin deposition, the method comprising: a) administering a composition comprising fibrin-targeted microbubbles described herein to a patient suspected or at risk of having a fibrin-associated condition or disorder wherein the fibrin-targeted microbubbles bind to fibrin at the site of fibrin deposition in the patient; and b) performing ultrasound imaging to detect the fibrin-targeted microbubbles bound at the site of fibrin deposition in the patient. Such imaging may be used to detect a fibrin-associated condition or disorder including, but not limited to, adhesions such as surgically-induced adhesions, adhesions caused by traumatic injury, abdominal adhesions, adhesions associated with adhesive capsulitis, pelvic adhesions, peridural adhesions, peritendinous adhesions, and pericardial adhesions; and plaques associated with thrombosis and atherosclerotic cardiovascular diseases. In certain embodiments, an adhesion (e.g., an adhesion caused by surgery, traumatic injury, or disease) or atherosclerotic plaque is present at the site of fibrin deposition in the patient. In some embodiments, the adhesion is an abdominal cancer surgery adhesion.

In another aspect, the invention includes a method of detecting an adhesion, the method comprising: a) administering a composition comprising a collection of fibrin-targeted microbubbles to a patient suspected or at risk of having an adhesion, under conditions wherein the fibrin-targeted microbubble binds to fibrin on any adhesions, if present, in the patient; and b) performing ultrasound imaging to detect the fibrin-targeted microbubbles bound to any adhesions, if present, in the patient.

In another aspect, the invention includes a method of treating an adhesion, the method comprising: a) administering a composition comprising fibrin-targeted microbubbles to a patient having an adhesion, under conditions wherein a fibrin-targeted microbubble binds to fibrin on the adhesion in the patient; and b) exposing the patient to ultrasound in the vicinity of the adhesion to break up the adhesion. For example, the methods of the invention can be used for treating surgically induced adhesions and adhesion-associated disorders such as small bowel obstruction, post-surgical morbidity, including chronic abdominal or pelvic pain, infertility in women, and potentially fatal intestinal obstructions. Adhesions that may be treated by the methods of the invention include surgically-induced adhesions, adhesions caused by traumatic injury, abdominal adhesions, adhesions associated with adhesive capsulitis, pelvic adhesions, peridural adhesions, peritendinous adhesions, and pericardial adhesions, as well as plaques associated with thrombosis and atherosclerotic cardiovascular diseases. In some embodiments, the adhesion is an abdominal cancer surgery adhesion. In certain embodiments, the invention includes a method of treating an abdominal cancer surgery adhesion, the method comprising: a) administering a composition comprising fibrin-targeted microbubbles to a patient having an abdominal cancer surgery adhesion, under conditions wherein a fibrin-targeted microbubble binds to fibrin on the adhesion in the patient; and b) exposing the patient to ultrasound in the vicinity of the adhesion to break up the adhesion.

In another embodiment, the fibrin-targeted microbubble further comprises an agent encapsulated within the polymerized lipid shell, such as a therapeutic agent (e.g., small molecule drug or therapeutic peptide, protein, or antibody) or imaging agent, wherein the agent is delivered to an adhesion by the fibrin-targeted microbubble.

In another aspect, the invention includes a method of making polydisperse microbubbles, the method comprising: a) conjugating a fibrin-targeting agent to a non-polymerizable lipid; b) mixing at least one polymerizable lipid with at least one non-polymerizable lipid to form a lipid mixture, wherein at least one non-polymerizable lipid is conjugated to the fibrin-targeting agent; c) forming liposomes in water comprising the lipid mixture; d) adding a gas; e) sonicating the liposomes in the presence of the gas to form polydisperse, gas-filled microbubbles; and f) exposing the microbubbles to ultraviolet light to polymerize the polymerizable lipid to form polydisperse polymerized shell lipid microbubbles. In certain embodiments, the polymerizable lipid is polymerized by diacetylene polymerization. In certain embodiments, the microbubbles are exposed to ultraviolet light for at least 2 minutes to polymerize the polymerizable lipid. For example, the microbubbles may be exposed to ultraviolet light for 2-5 minutes to polymerize the polymerizable lipid, or any amount of time in this range such as 2, 2.5, 3, 3.5, 4, 4.5, or 5 minutes. In certain embodiments, the lipid mixture comprises at least about 5% polymerizable lipid. For example, the polymerizable lipid may range from about 5% to about 50% of the total lipid in the lipid mixture, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%. In certain embodiments, at least one non-polymerizable or at least one polymerizable lipid is PEGylated. For example, the PEGylated lipid may range from about 5% to about 50% of the total lipid in the lipid mixture, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%. In another embodiment, the non-polymerizable lipid is 1,2-distearoyl-sn-glycero-3-phosphocholine (DSPC), 1,2-distearoyl-sn-glycero-3-phosphoethanolamine-N-[methoxy(polyethylene glycol)-2000 (DSPE-PEG2000), or DSPE-PEG2000-biotin. In another embodiment, the polymerizable lipid is a diacetylenic lipid. In another embodiment, the fibrin-targeting agent is conjugated to the non-polymerizable lipid in the polymerized lipid shell. In another embodiment, the fibrin-targeting agent is a fibrin-binding peptide such as the CREKA peptide. An exemplary fibrin-targeted microbubble with the CREKA peptide comprises a polymerized lipid shell comprising 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide. In one embodiment, the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25. In certain embodiments, the gas is a heavy gas. Exemplary gases that may be used include a perfluorocarbon (e.g., decafluorobutane), nitrogen, or air. In another embodiment, the gas comprises at least two perfluorocarbons.

In another aspect, the invention includes a method of making monodisperse polymerized shell lipid microbubbles, the method comprising: a) conjugating a fibrin-targeting agent to a non-polymerizable lipid; b) mixing at least one polymerizable lipid with at least one non-polymerizable lipid to form a lipid mixture, wherein at least one non-polymerizable lipid is conjugated to the fibrin-targeting agent; c) using microfluidic flow focusing of the lipid mixture in the presence of a gas through an aperture to form microbubbles; d) exposing the microbubbles to ultraviolet light to polymerize the polymerizable lipid to produce monodisperse polymerized shell lipid microbubbles. In certain embodiments, the polymerizable lipid is polymerized by diacetylene polymerization. In certain embodiments, the microbubbles are exposed to ultraviolet light for at least 2 minutes to polymerize the polymerizable lipid. For example, the microbubbles may be exposed to ultraviolet light for 2-5 minutes to polymerize the polymerizable lipid, or any amount of time in this range such as 2, 2.5, 3, 3.5, 4, 4.5, or 5 minutes. In certain embodiments, the lipid mixture comprises at least about 5% polymerizable lipid. For example, the polymerizable lipid may range from about 5% to about 50% of the total lipid in the lipid mixture, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%. In certain embodiments, at least one non-polymerizable or at least one polymerizable lipid is PEGylated. For example, the PEGylated lipid may range from about 5% to about 50% of the total lipid in the lipid mixture, including any percentage within this range, such as 5%, 6%, 7%, 8%, 9%, 10%, 11%, 12%, 13%, 14%, 15%, 16%, 17%, 18%, 19%, 20%, 22%, 24%, 26%, 28%, 30%, 35%, 40%, 45%, or 50%. In another embodiment, the non-polymerizable lipid is 1,2-distearoyl-sn-glycero-3-phosphocholine (DSPC), 1,2-distearoyl-sn-glycero-3-phosphoethanolamine-N-[methoxy(polyethylene glycol)-2000 (DSPE-PEG2000), or DSPE-PEG2000-biotin. In another embodiment, the polymerizable lipid is a diacetylenic lipid. In another embodiment, the fibrin-targeting agent is conjugated to the non-polymerizable lipid in the polymerized lipid shell. In another embodiment, the fibrin-targeting agent is a fibrin-binding peptide such as a CREKA peptide. An exemplary fibrin-targeted microbubble with the CREKA peptide comprises a polymerized lipid shell comprising 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide. In one embodiment, the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25. In certain embodiments, the gas is a heavy gas. Exemplary gases that may be used include a perfluorocarbon (e.g., decafluorobutane), nitrogen, or air. In another embodiment, the gas comprises at least two perfluorocarbons.

These and other embodiments of the subject invention will readily occur to those of skill in the art in view of the disclosure herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 shows polymerized shell microbubble stability after UV cross-linking. The change in diameter of the mono- and poly-disperse PSM over 6 days. Size data was obtained from bright field images.

FIGS. 2A-2B show PSM culture plate data. FIG. 2A shows the change in fluorescence in fibrin- or fibrinogen-coated plates after treatment with CREKA-targeted PSMs and a wash step. FIG. 2B shows a determination of binding affinity of CREKA-targeted and non-targeted PSMs to collagen-coated cell culture plates and cultured LP-9 cells coated with fibrin. Samples of PSM treatment solution after a 30-minute incubation, and PSM fluorescence was compared with a sample of unbound PSMs to determine % bound in each condition. Because % bound is calculated based on a negative control, values >100% are possible.

FIG. 3 shows the midline incision made in anaesthetized animals. Sutures are used to tie off 3 buttons in the peritoneal wall on both sides of the peritoneum. Positive control animals have their peritoneal wall cut and scraped instead to form ablations. Negative control animals receive no surgery. PSMs were injected 24 hours after surgery and imaged with ultrasound (US) 1, 2 and 24 hours after injection. Animals were then euthanized, reopened, and the presence or absence of adhesions was recorded for each button and ablation.

FIGS. 4A-4D show the results of in vivo experiments: FIG. 4A shows the experimental conditions and scoring of results for the in vivo experiment. Targeted polydisperse PSMs enabled accurate diagnosis of adhesions with ultrasound (US) while non-targeted polydisperse PSMs gave random results (analyzed by one-way ANOVA, p<0.05). FIG. 4B shows PSMs visible via US in rat peritoneum with no buttons or adhesions present. FIG. 4C shows an US image of non-targeted PSMs showing random spots. These can sometimes form shapes resembling adhesions, but there was no correlation between these shapes and actual adhesions. FIG. 4D shows the ultrasound image of CREKA-targeted PSMs showing adhesion-associated spots (in circles). (Dark shapes (indicated by arrows) are large peritoneal structures like the bowel.)

FIGS. 5A-5D show images of the post-treatment peritoneal wall: FIG. 5A shows a post-experiment image showing adhesions (arrows) linking two buttons to other tissue. FIG. 5B shows the buttons (arrows) in an animal treated with targeted monodisperse PSMs appear inflamed, but no adhesions are present. FIG. 5C shows a massive adhesion (arrow) spanning all 3 ablations is visible in a positive control animal treated with non-targeted PSMs. FIG. 5D shows that in a positive control animal treated with targeted monodisperse PSMs, the ablations are clearly visible (arrows) but no adhesions are present.

### DETAILED DESCRIPTION OF THE INVENTION

Targeted contrast ultrasound offers the potential to target specific molecular markers in the body, revealing information about molecular makeup in addition to structure. In particular, the inventors have developed fibrin-targeted polymerized shell lipid microbubbles (PSMs) in both monodisperse and polydisperse formulations. The inventors have further shown that such microbubbles can be used diagnostically in ultrasound imaging of adhesions and are also useful therapeutically in breaking up abdominal adhesions (see Examples).

The practice of the present invention will employ, unless otherwise indicated, conventional methods of medicine, pharmacology, chemistry, biochemistry, molecular biology and recombinant DNA techniques and immunology, within the skill of the art. Such techniques are explained fully in the literature. See, e.g., C. M. Rumack, S. R. Wilson, J. W. Charboneau, and D. Levine Diagnostic Ultrasound (Mosby, 4^{th} edition, 2016); A. B. Wolbarst et al. Medical Imaging: Essentials for Physicians (Wiley-Blackwell, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

Unless otherwise explained, all technical and scientific terms used herein have the same meaning as commonly understood by one of ordinary skill in the art to which this disclosure belongs. All references cited herein are all incorporated by reference herein in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention.

Other than in the operating examples, or where otherwise indicated, all numbers expressing quantities of ingredients or reaction conditions used herein should be understood as modified in all instances by the term “about” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

The term “animal” or “animal subject” or “individual” or “subject” or “patient” as used herein includes humans as well as other mammals. In some embodiments, the subject is a human, a monkey, a dog, a pig, a bovine, a rabbit, a guinea pig, and/or a rodent. In some embodiments, the methods involve the administration of one or more microbubbles for the treatment of one or more conditions. Combinations of agents can be used to treat one condition or multiple conditions or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying condition being treated. Also, a therapeutic benefit is achieved with the eradication or amelioration of one or more of the physiological symptoms associated with the underlying condition such that an improvement is observed in the patient, notwithstanding that the patient may still be afflicted with the underlying condition. For prophylactic benefit, the compositions may be administered to a patient at risk of developing a particular disease, or to a patient reporting one or more of the physiological symptoms of a disease, even though a diagnosis of this disease may not have been made.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

A “fibrin-associated condition or disorder” refers to any condition or disorder that causes fibrin deposition in tissue. Fibrin-associated conditions and disorders include, but are not limited to, adhesions such as surgically-induced adhesions, adhesions caused by traumatic injury, abdominal adhesions, adhesions associated with adhesive capsulitis, pelvic adhesions, peridural adhesions, peritendinous adhesions, and pericardial adhesions; and plaques associated with thrombosis and atherosclerotic cardiovascular diseases.

By “therapeutically effective dose or amount” of a fibrin-targeted microbubble is intended an amount that, when administered as described herein, brings about a positive therapeutic response in treatment of a fibrin-associated condition or disorder, such as an amount sufficient to diminish or break up an adhesion or plaque. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “antibody” encompasses monoclonal and polyclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fᵥ molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., fibrin) refers to a binding reaction that is determinative of the presence of the antigen in a heterogeneous population of proteins and other biologics. Thus, under designated immunoassay conditions, the specified antibodies bind to a particular antigen at least two times the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

### Fibrin-Targeted Microbubbles

In one aspect, the present invention provides fibrin-targeted microbubbles. The term microbubbles refers to vesicles which are generally characterized by the presence of one or more membranes or walls or shells surrounding an internal void that is filled with a gas or precursor thereto. In some embodiments, the shell of the microbubble comprises one or more lipids. The term lipids includes agents exhibiting amphipathic characteristics causing them to spontaneously adopt an organized structure in water wherein the hydrophobic portion of the molecule is sequestered away from the aqueous phase. In some embodiments, the microbubbles comprise polymerizable lipids. In some embodiments, the microbubbles comprise one or more lipids, at least one of which is polymerizable. In some embodiments the microbubbles comprise one or more gases inside a lipid shell. In addition, the microbubbles further comprise a fibrin-targeting agent attached to the lipid shell either directly or indirectly through a linker. The microbubbles may optionally also contain therapeutic agents, and/or other functional molecules, either attached to the lipid shell or enclosed inside the microbubble. The microbubbles of the invention may also include any other materials or combination thereof known to those skilled in the art as suitable for microbubble construction.

### Lipids

In one aspect, the microbubbles of the invention comprise one or more lipids. The lipids used may be of natural and/or synthetic origin. Such lipids include, but are not limited to, fatty acids, lysolipids, dipalmitoylphosphatidylcholine, phosphatidylcholine, phosphatidic acid, sphingomyelin, cholesterol, cholesterol hemisuccinate, tocopherol hemisuccinate, phosphatidylethanolamine, phosphatidyl-inositol, lysolipids, sphingomyelin, glycosphingolipids, glucolipids, glycolipids, sulphatides, lipids with ether and ester-linked fatty acids, diacetyl phosphate, stearylamine, distearoylphosphatidylcholine, phosphatidylserine, sphingomyelin, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galactopyranoside, 6-(5-cholesten-3-β-yloxy)hexyl-6-amino-6-deoxyl-1-thio-α-D-mannopyranoside, dibehenoyl-phosphatidylcholine, dimyristoylphosphatidylcholine, dilauroylphosphatidylcholine, and dioleoyl-phosphatidylcholine, and/or combinations thereof.

In some embodiments, the microbubbles of the invention comprise one or more polymerizable lipids. Examples of polymerizable lipids include but are not limited to, diacetylene lipids such as diyne PC (1,2-bis(10,12-tricosadiynoyl)-sn-glycero-3-phosphocholine) and diynePE (1,2-bis(10,12-tricosadiynoyl)-sn-glycero-3-phosphoethanolamine). The polymerizable lipids that can be used in the present invention may also include those described in U.S. Pat. Nos. 5,512,294 and 6,132,764, and US publication No. 2010/0111840, herein incorporated by reference in their entireties. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% polymerizable lipids. In some embodiments, the microbubbles of the invention comprise at least 25% polymerizable lipids. In some embodiments, the microbubbles of the invention comprise at least 50% polymerizable lipids. In some embodiments, the polymerizable lipid may comprise a polymerizable group attached to a lipid molecule. The microbubbles may also contain lipids that are not polymerizable, lipids conjugated to a functional moiety (such as a targeting agent or a therapeutic agent), and lipids with a positive, negative, or neutral charge.

In some embodiments, the microbubbles of the invention comprise one or more neutral phospholipids. Examples of neutral phospholipids include, but are not limited to, hydrogenated phosphatidyl choline (HSPC), dipalmitoyl-, distearoyl- and diarachidoyl phosphatidylcholine (DPPC, DSPC, DAPC). In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of neutral phospholipids. In some embodiments, the microbubbles of the invention comprise at least 10% neutral phospholipids. In some embodiments, the microbubbles of the invention comprise at least 30% neutral phospholipids. In some embodiments, the microbubbles of the invention comprise at least 45% neutral phospholipids.

In some embodiments, the microbubbles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG). In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% negatively charged phospholipids. In some embodiments, the microbubbles of the invention comprise at least 2% negatively charged phospholipids. In some embodiments, the microbubbles of the invention comprise at least 5% negatively charged phospholipids. In some embodiments, the microbubbles of the invention comprise at least 10% negatively charged phospholipids. In some embodiments, the microbubbles of the invention comprise at least 25% negatively charged phospholipids. In some embodiments, the microbubbles of the invention comprise at least 30% negatively charged phospholipids.

In some embodiments, the microbubbles of the invention comprise one or more reactive phospholipids. Examples of reactive phospholipids include, but are not limited to, phosphatidyl ethanolamine derivatives coupled to a polyethyleneglycol, a biotinyl, a glutaryl, a caproyl, a maleimide, a sulfhydral, a pyridinal disulfide or a succinyl amine. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% reactive phospholipids. In some embodiments, the microbubbles of the invention comprise at least 2% reactive phospholipids. In some embodiments, the microbubbles of the invention comprise at least 5% reactive phospholipids. In some embodiments, the microbubbles of the invention comprise at least 10% reactive phospholipids. In some embodiments, the microbubbles of the invention comprise at least 25% reactive phospholipids. In some embodiments, the microbubbles of the invention comprise at least 30% reactive phospholipids.

In some embodiments, the microbubbles of the invention comprise one or more lipids and phospholipids such as soy lecithin, partially refined lecithin, hydrogenated phospholipids, lysophosphate, phosphatidylcholine, phosphatidylethanolamine, phosphatidylserine, phosphatidylinositol, cardiolipin, sphingolipids, gangliosides, cerebrosides, ceramides, other esters analogue of phosphatidylcholine (PAF, lysoPAF). In some embodiments, the microbubbles of the invention comprise one or more synthetic phospholipids such as L-α-lecithin (dilauroylphosphatidylcholine, dipalmitoylphosphatidylcholine, dilinoloylphosphatidylcholine, distearoylphosphatidylcholine, diarachidoylphosphatidylcholine); phosphatidylethanolamine derivatives, such as 1,2-diacyl-sn-glycero-3-phosphoethanolamine, 1-acyl-2-acyl-sn-glycero-3-phosphoethanolamine, dinitrophenyl- and dinitrophenylamino caproylphosphatidylethanolamine, 1,2-diacyl-sn-glycero-3-phosphoethanolamine-N-polyethylene glycol (PEG-PE), N-biotinyl-PE, N-caproylamine PE, N-dodecylamine-PE, N-MPB-PE, N-PDD-PE, N-succinyl-PE, N-glutaryl-PE; di-acetylenic lipids; phosphatidic acids (1,2-diacyl-sn-glycero-3-phosphate salt, 1-acyl-2-acyl-sn-glycero-3-phosphate sodium salt; phosphatidylserine such as 1,2-diacyl-snglycero-3-[phospho-L-serine] sodium salt, 1-acyl-2-acyl-sn-glycero-3-[phospho-L-serine] sodium salt, lysophosphatidic acid; cationic lipids such as 1,2-diacyl-3-trimethylammoniumpropane (TAP), 1,2-diacyl-3-dimethylammoniumpropane (DAP), N-[1-(2,3-dioleoyloxy) propyl-N,N′,N″-trimethylammonium chloride (DOTMA).

In some embodiments, the microbubbles of the invention comprise one or more lipids suitable for click chemistry, such as those containing azide and alkyne groups. In some embodiments, the microbubbles of the invention comprise one or more phospholipids with various headgroups such as phosphatidylethanol, phosphatidylpropanol and phosphatidylbutanol, phosphatidylethanolamine-N-monomethyl, 1,2-disteraoyl(dibromo)-sn-glycero-3-phosphocoline. In some embodiments, the microbubbles of the invention comprise one or more phospholipids with partially or fully fluorinated cholesterol or cholesterol derivatives can be used in place of an uncharged lipid, as generally known to a person skilled in the art.

The surface of a microbubble may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as a therapeutic entity. Any species of lipid may be used, with the sole proviso that the lipid or combination of lipids and associated materials incorporated within the lipid matrix should form a monolayer phase under physiologically relevant conditions. As one skilled in the art will recognize, the composition of the microbubble may be altered to modulate the biodistribution and clearance properties of the resulting microbubbles.

Other useful lipids or combinations thereof apparent to those skilled in the art which are in keeping with the spirit of the present invention are also encompassed by the present invention. For example, carbohydrates bearing lipids may be employed for in vivo targeting as described in U.S. Pat. No. 4,310,505.

In some embodiments, the hydrophobic tail groups of the polymerizable lipids are derivatized with polymerizable groups, such as diacetylene groups, which irreversibly cross-link, or polymerize, when exposed to ultraviolet light or other radical, anionic or cationic, initiating species, while maintaining the distribution of functional groups at the surface of the microbubble. The resulting polymerized microbubble particle is stabilized against fusion with cell membranes or other microbubbles and stabilized towards enzymatic degradation. The size of the polymerized microbubbles can be controlled by the methods described herein, but also by other methods known to those skilled in the art, for example, by extrusion.

Polymerized microbubbles may comprise not only polymerizable lipids but also saturated and non-alkyne, unsaturated lipids. The polymerized microbubbles may comprise a mixture of lipids, which provide different functional groups on the hydrophilic exposed surface. For example, some hydrophilic head groups can have functional surface groups, for example, biotin, amines, cyano, carboxylic acids, isothiocyanates, thiols, disulfides, α-halocarbonyl compounds, α,β-unsaturated carbonyl compounds and alkyl hydrazines. These groups can be used for attachment of fibrin-binding targeting agents, such as fibrin-specific antibodies, ligands, proteins, peptides, carbohydrates, aptamers, or antibody mimetics, or combinations thereof for specific targeting pf microbubbles to fibrin, and/or for attachment of therapeutic agents, such as drugs, therapeutic proteins or antibodies, nucleic acids encoding genes with therapeutic effects, or radioactive isotopes. Other head groups may have an attached or encapsulated therapeutic agent, such as, for example, antibodies, hormones and drugs for interaction with a biological site at or near the site of fibrin deposition to which the polymerized microbubble particle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphyrin chelate and cyclohexane-1,2-diamino-N,N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

Large numbers of therapeutic agents may be attached to a single polymerized microbubble that may also bear from several to about one thousand targeting agents for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and metastatic cancer. For example, multi-valent sialyl Lewis X derivatized microbubbles can be used to block neutrophil binding, and antibodies against VCAM-1 on polymerized microbubbles can be used to block lymphocyte binding, e.g. T-cells.

The polymerized microbubble particle can also contain groups to control nonspecific adhesion and reticuloendothelial system uptake. For example, PEGylation of liposomes has been shown to prolong circulation lifetimes; see International Patent Application WO 90/04384.

The component lipids of polymerized microbubbles can be purified and characterized individually using standard, known techniques and then combined in controlled fashion to produce the final particle. The polymerized microbubbles can be constructed to mimic native cell membranes or present functionality, such as ethylene glycol derivatives, that can reduce their potential immunogenicity. Additionally, the polymerized microbubbles have a well-defined monolayer structure that can be characterized by known physical techniques such as transmission electron microscopy and atomic force microscopy.

### Gases

In one aspect the invention provides gas filled microbubbles. In some embodiments the microbubbles comprise one or more gases inside a lipid shell. In some embodiments, the lipid shell comprises one or more polymerizable lipids. In some embodiments, the invention provides gas filled microbubbles substantially devoid of liquid in the interior. In some embodiments, the microbubbles are at least about 90% devoid of liquid, at least about 95% devoid of liquid, or about 100% devoid of liquid.

The microbubbles may contain any combination of gases suitable for the diagnostic or therapeutic method desired. For example, various biocompatible gases such as air, nitrogen, carbon dioxide, oxygen, argon, xenon, neon, helium, and/or combinations thereof may be employed. Other suitable gases will be apparent to those skilled in the art, the gas chosen being only limited by the proposed application of the microbubbles.

In some embodiments, the microbubbles contain gases with high molecular weight and size. In some embodiments, the microbubbles contain fluorinated gases, fluorocarbon gases, and perfluorocarbon gases. In some embodiments, the perfluorocarbon gases include perfluoropropane, perfluorobutane, perfluorocyclobutane, perfluoromethane, perfluoroethane and perfluoropentane, especially perfluoropropane. In some embodiments, the perfluorocarbon gases have less than six carbon atoms. Gases that may be incorporated into the microbubbles include but are not limited to: SF₆, CF₄, C₂F₆, C₃F₆, C₃F₈ C₄F₆, C₄F₅, C₄F₁₀, C₅F₁₀, C₅F₁₂, C₆F₁₂, (1- trifluoromethyl), propane (2-trifluoromethyl)-1,1,1,3,3,3 hexafluoro, and butane (2-trifluoromethyl)-1,1,1,3,3,3,4,4,4 nonafluor, air, oxygen, nitrogen, carbon dioxide, noble gases, vaporized therapeutic compounds, and mixtures thereof. The halogenated versions of hydrocarbons, where other halogens are used to replace F (e.g., Cl, Br, I) would also be useful.

In some embodiments, microbubbles containing gases with high molecular weights and sizes are used for ultrasound imaging purposes.

In some embodiments, innocuous, low boiling liquids which vaporize at body temperature or by the action of remotely applied energy pulses, like C₆F₁₄, are also usable as a volatile confinable microbubble component in the present invention. In some embodiments, the confined gases may be at atmospheric pressure or under pressures higher or lower than atmospheric; for instance, the confined gases may be at pressures equal to the hydrostatic pressure of the carrier liquid holding the gas filled microspheres.

In some embodiments, the microbubbles comprise mixtures of these gases, e.g., mixtures of perfluorocarbons with other perfluorocarbons and mixtures of perfluorocarbons with other gases, such as air, N₂, O₂, or He. The first gas and the second gas can be respectively present in a molar ratio of about 1:100, 1:75, 1:50, 1:30, 1:20, 1:10, 1:5 or 1:1 to about 1000:1, 500:1, 250:1, 100:1, 75:1, 50:1, 10:1 or 5:1.

### Fibrin Targeting of Microbubbles

In some embodiments, the microbubbles of the invention comprise a fibrin targeting agent attached to the polymerized lipid shell of the microbubbles. The term fibrin targeting agent includes a molecule, macromolecule, or molecular assembly which binds specifically to fibrin (e.g., fibrin in adhesions or plaques). Any biologically compatible, natural or artificial molecule that selectively binds to fibrin may be utilized as a fibrin-targeting agent. Examples of fibrin targeting agents include, but are not limited to, peptides, proteins, antibodies, antibody fragments and other antibody-derived molecules which retain specific binding, such as Fab, F(ab′)₂, Fᵥ, diabodies and scFv derived from antibodies); antibody mimetics, and aptamers that specifically bind to fibrin.

In some embodiments, the fibrin targeting agent is a peptide which specifically binds to fibrin. An exemplary fibrin-binding peptide is the CREKA peptide comprising the amino acid sequence: cysteine-arginine-glutamic acid-lysine-alanine (SEQ ID NO:1) and CPT-11. Fibrin-targeted microbubbles comprising the CREKA peptide can be prepared, for example, from 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC) hydrogenated soy (soy PC) and 5′-hydroxy-3′-oxypentyl-10-12-pentacosadiynamide (h-PEG1-PCDA) in a molar ratio of 15:60:25 (see Example 2).

Alternatively, an anti-fibrin antibody may be attached to microbubbles as a fibrin-targeting agent. Any type of antibody may be used, including polyclonal and monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fᵥ molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule (i.e., specifically binds to fibrin). Anti-fibrin antibodies are commercially available from a variety of sources, including, for example, Creative Diagnostics (Shirley, N.Y.), LifeSpan Biosciences Inc. (Seattle, Wash.), and GeneTex (Irvine, Calif.).

In other embodiments, the fibrin-targeting agent comprises an aptamer that specifically binds to fibrin. Any type of aptamer may be used, including a DNA, RNA, xeno-nucleic acid (XNA), or peptide aptamer that specifically binds to fibrin. Such aptamers can be identified, for example, by screening a combinatorial library. Nucleic acid aptamers (e.g., DNA or RNA aptamers) that bind selectively to fibrin can be produced by carrying out repeated rounds of in vitro selection or systematic evolution of ligands by exponential enrichment (SELEX). Peptide aptamers that bind to fibrin may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEX for Theranostics (W. Tan, X. Fang eds., Springer, 2015), Cox et al. (2001) Bioorg. Med. Chem. 9(10):2525-2531; Cox et al. (2002) Nucleic Acids Res. 30(20): e108, Kenan et al. (1999) Methods Mol Biol. 118:217-231; Platella et al. (2016) Biochim. Biophys. Acta Nov 16 pii: S0304-4165(16)30447-0, and Lyu et al. (2016) Theranostics 6(9):1440-1452; herein incorporated by reference in their entireties.

In yet other embodiment, the fibrin-targeting agent comprises an antibody mimetic that specifically binds to fibrin. Any type of antibody mimetic may be used, including, but not limited to, affibody molecules (Nygren (2008) FEBS J. 275 (11):2668-2676), affilins (Ebersbach et al. (2007) J. Mol. Biol. 372 (1):172-185), affimers (Johnson et al. (2012) Anal. Chem. 84 (15):6553-6560), affitins (Krehenbrink et al. (2008) J. Mol. Biol. 383 (5):1058-1068), alphabodies (Desmet et al. (2014) Nature Communications 5:5237), anticalins (Skerra (2008) FEBS J. 275 (11):2677-2683), avimers (Silverman et al. (2005) Nat. Biotechnol. 23 (12):1556-1561), darpins (Stumpp et al. (2008) Drug Discov. Today 13 (15-16):695-701), fynomers (Grabulovski et al. (2007) J. Biol. Chem. 282 (5):3196-3204), and monobodies (Koide et al. (2007) Methods Mol. Biol. 352:95-109).

The fibrin targeting agents can be attached to microbubbles using any feasible method known in the art such as carbodiimide, maleimide, disulfide, or biotin-streptavidin coupling. In one embodiment, the attachment is by covalent means. In another embodiment, the attachment is by non-covalent means. For example, a fibrin-target agent (e.g., peptide, antibody, or aptamer that specifically binds to fibrin) may be biotinylated and attached to an avidin or streptavidin coated microbubble.

Imaging and Treatment with Fibrin-Targeted Microbubbles

The fibrin-targeting agents, which are attached to the lipid shells of the microbubbles, serve to selectively concentrate microbubbles in regions where fibrin is present in tissue, and can be used for imaging or therapeutic treatment of patients having fibrin-associated conditions or disorders. The fibrin targeting of microbubbles selectively enhances imaging of and/or therapeutic delivery of therapeutic agents to sites of fibrin deposition in tissue (e.g., adhesions or plaques). In certain embodiments, fibrin-targeted microbubbles are used for the classification, diagnosis, prognosis, determination of a condition stage, determination of response to treatment, or monitoring or predicting outcome of a fibrin-associated condition or disorder.

Fibrin-associated conditions and disorders include any condition or disorder that causes fibrin deposition in tissue such as, but not limited to, adhesions, including surgically-induced adhesions, adhesions caused by traumatic injury, abdominal adhesions, adhesions associated with adhesive capsulitis, pelvic adhesions, peridural adhesions, peritendinous adhesions, and pericardial adhesions; and plaques associated with thrombosis and atherosclerotic cardiovascular diseases. In some embodiments, the adhesion is an abdominal cancer surgery adhesion.

Fibrin-targeting of microbubbles is particularly suitable for diagnostic imaging, for example, to determine locations of adhesions or atherosclerotic plaques. Preferably, a detectably effective amount of the fibrin-targeted microbubbles is administered to a subject; that is, an amount that is sufficient to yield an acceptable image using the ultrasound imaging equipment that is available for clinical use. A detectably effective amount of the fibrin-targeted microbubbles may be administered in more than one injection if needed. The detectably effective amount of the fibrin-targeted microbubbles needed for ultrasound imaging of an individual may vary according to factors such as the degree of binding to an area of fibrin deposition in tissue (e.g., adhesion or plaque), the age, sex, and weight of the individual, and the particular ultrasound imaging device used. Optimization of such factors is within the level of skill in the art.

Once administered, microbubbles may be monitored by any suitable means known in the art. In some embodiments, the microbubbles may be monitored and/or detected by ultrasonic imaging means, or by MRI or radiography if the formulation includes agents for such imaging. Ultrasound imaging techniques including but not limited to harmonic imaging, subharmonic imaging, pulse inversion mode imaging, passive cavitation mapping, and any version of Doppler mode imaging may be employed to visualize microbubble activity. The ultrasonic irradiation may be carried out by a modified echography probe adapted to simultaneously monitor the echo signal and thereby provide an image of the irradiated site. The monitoring signal may be in the range of 20 kHz to 40 MHz. In some embodiments, the monitoring signal is in the range of 1-3 MHz. In an exemplary embodiment, microbubbles are visualized using a portable diagnostic ultrasound system (Terason 2000, Teratech, Burlington, Mass.) along with a 5-10 MHz clinical ultrasound transducer (L10-5, Terason, Burlington, Mass.).

In some embodiments, the enhanced stability of the PSMs of the present invention allows visualization of regions further from the administration site and for greater periods of time. Among other assays, PSMs may be used to visualize the vascular system. In visualizing a patient's vasculature, blood flow may be measured, as will be well understood by those skilled in the art. Example of detection methods that can be used in the methods herein are described in US publications U.S. Pat. Nos. 5,769,080; 5,209,720; 6,132,764; 6,132,764; and 20100111840, incorporated by reference herein in their entirety.

Mechanical Treatment with Fibrin-Targeted Microbubbles

It is well known that, under the influence of externally imposed ultrasound, the oscillations of bubbles, whether stable volumetric oscillations, stable shape oscillations, or transient large expansions followed by inertial collapses, can impart mechanical stress and result in any of several bioeffects, including but not limited to mechanical damage including cell lysis (e.g., so called ‘histotripsy’ ‘sonothrombolysis’), thermal damage (so-called ‘focused ultrasound surgery’, ‘high-intensity focused ultrasound’, gene transfection, permeabilization (so-called ‘sonoporation’ and ‘blood-brain-barrier disruption’, to tissues. In any of these situations, by incorporating molecular targeting in these micro bubbles, a theranostic action can be achieved to image and destroy the targeted tissue. In particular, short (on the order of 10's of cycles of the ultrasound) pulses of ultrasound which induce no ultrasound heating effect and minimal radiation force effects result in purely mechanical effects when bubbles are present.

The present discovery and invention belong to this latter class of purely mechanical bubble bioeffects. By incorporating molecular agents described above in the “Summary of the invention” section, as well as in the “Fibrin-Targeted Microbubbles” and the “Fibrin Targeting of Microbubbles” sections, bubbles injected into the body will adhere to fibrin such as is found in newly-formed adhesions. The present invention shows that only modest, non-inertial bubble oscillations are required to break up newly-formed surgical adhesions under the influence of short-pulse diagnostic ultrasound from a standard clinical ultrasound scanner. Details of a preferred embodiment are described below in section “Examples”. The phrase ‘modest, non-inertial’ above will be understood to include stable volumetric as well as stable shape oscillations, but exclude transient inertia-controlled collapses, whether or not those inertial collapses result in re-entrant jet formation. It is, of course, well known that transient inertial oscillations result in mechanical damage and are capable of breaking up adhesions, but transient inertial oscillations require transducers and driving systems capable of achieving higher acoustic pressures than a clinical ultrasound scanner is capable of. The present invention utilizes only the acoustics provided by clinical diagnostic ultrasound scanners in so-called ‘B-mode’, the most common imaging modality. Other acoustic modalities offered by clinical ultrasound scanners, such as M-mode, Doppler and Power Doppler, are not needed but will also prove efficacious.

### Incorporation of Multi-Modality Contrast Agents

In some embodiments, the microbubbles described herein may also contain substances to enhance imaging, e.g., for diagnostics or to visualize treatment during drug delivery. Any suitable contrast agent known in the art can be incorporated into the microbubbles. These can include paramagnetic gases, such as atmospheric air, which contains traces of oxygen 17, or paramagnetic ions such as Mn⁺², Gd⁺², and Fe⁺³, to be used as susceptibility contrast agents for magnetic resonance imaging. Microbubbles may contain radio-opaque metal ions, such as iodine, barium, bromine, or tungsten, for use as x-ray contrast agents. Fibrin-targeted microbubbles may also be associated with other ultrasound contrast enhancing agents, such as SHU-454 or other microbubbles.

### Adjuvant Therapeutic Agents and Drug Delivery

Many different drugs or other therapeutic agents can be linked to or encapsulated within microbubbles. As some non-limiting examples, microbubbles may be used as carriers for delivery of antiangiogenics to treat tumors, anti-atherosclerotic drugs to treat plaques in the vasculature, or local anesthetics to anesthetize a specific area region of interest. Because microbubbles can be extremely stable, they may also be used for slow-release of therapeutics.

In some embodiments, a therapeutic agent may be incorporated into the microbubbles. A variety of drugs and other bioactive compounds may be incorporated into the microbubbles, including antineoplastic agents, blood products, biological response modifiers, anti-fungals, hormones, vitamins, peptides, proteins, antibodies, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, monoclonal antibodies, genetic material, and prodrugs.

In some embodiments, some of the bioactive compounds that may be incorporated into the microbubbles include genetic material such as nucleic acids, RNA, and DNA, of either natural or synthetic origin, including recombinant RNA and DNA and antisense RNA and DNA, genes carried on expression vectors such as plasmids, phagemids, cosmids, yeast artificial chromosomes (YACs), and defective or “helper” viruses, antigene nucleic acids, both single and double stranded RNA and DNA and analogs thereof; hormone products such as vasopressin, oxytocin, progestins, estrogens and antiestrogens and their derivatives, glucagon, and thyroid agents such as iodine products and anti-thyroid agents; biological response modifiers such as muramyldipeptide, muramyltripeptide, microbial cell wall components, lymphokines (e.g., bacterial endotoxins such as lipopolysaccharide, macrophage activation factor), subunits of bacteria (such as Mycobacteria, Corynebacteria), the synthetic dipeptide N-acetyl-muramyl-L-alanyl-Disoglutamine; cardiovascular products such as chelating agents and mercurial diuretics and cardiac glycosides; blood products such as parenteral iron, hemin, hematoporphyrins and their derivatives; respiratory products such as xanthine derivatives (theophylline & aminophylline); anti-infectives such as aminoglycosides, antifungals (amphotericin, ketoconazole, nystatin, griseofulvin, flucytosine (5-fc), miconazole, amphotericin B, ricin, and 13-lactam antibiotics (e.g., sulfazecin)), antibiotics such as penicillins, actinomycins and cephalosporins, antiviral agents such as Zidovudine, Ribavirin, Amantadine, Vidarabine, and Acyclovir, anti-helmintics, antimalarials, and antituberculous drugs; biologicals such as immune serums, antitoxins and antivenins, rabies prophylaxis products, bacterial vaccines, viral vaccines, toxoids; antineoplastics such as nitrosureas, hydroxyurea, procarbazine, Dacarbazine, mitotane, nitrogen mustards, antimetabolites (fluorouracil), platinum compounds (e.g., spiroplatin, cisplatin, and carboplatin), methotrexate, adriamycin, taxol, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), teniposide (VM-26), vinblastine sulfate (VLB), vincristine sulfate, bleomycin, bleomycin sulfate, methotrexate, adriamycin, arabinosyl, and alkylated derivatives of metallocene dihalides; mitotic inhibitors such as Etoposide and the Vinca alkaloids, radiopharmaceuticals such as radioactive iodine and phosphorus products; as well as interferons (Interferon α-2a and α-2b), Asparaginase and cyclosporins.

The bioactives may be incorporated into microbubbles singly or in combination with each other or with additional substances aimed to increase bioactive efficacy, such as adjuvants. Bioactives may be attached (covalently, such as by ester, substituted ester, anhydride, carbohydrate, polylactide, or substituted anhydride bonds, or non-covalently, such as by streptavidin linkages or ionic binding) to the surface of the microbubble directly to the lipids or to a moiety conjugated to the lipids, incorporated directly into the lipid membrane, or included in the interior of the microbubble, preferably in a vapor state.

Targeting also enhances local administration of toxic substances which, if not targeted, could (and would) otherwise cause significant secondary effects to other organs; such drugs include for instance Amphotericin B or NSAID's or drugs whose administration is required over prolonged periods such as Dexamethasone, insulin, vitamin E, etc. The method is also suitable for administration of thrombolytic agents such as urokinase or streptokinase, or antitumoral compounds such as Taxol etc.

Prodrugs and otherwise non-active agents may also be incorporated into microbubbles with an activator, such as a protease that removes an inactivating peptide, such that the agent and the activator are separated until the microbubble is dissolved. Alternatively, the agent and the activator may be incorporated into different populations of microbubbles and targeted to the same location so that the agent is selectively activated only at the target site.

In some embodiments, the invention provides microbubbles comprising a therapeutic agent with a ratio by weight of the therapeutic agent to lipid in the microbubble of about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. In some embodiments, the ratio by weight of therapeutic agent to lipid is 1:2. In some embodiments, the ratio by weight of therapeutic agent to lipid is 1:1. In some embodiments, the therapeutic agent is in an oil:drug phase on the outer edge of the gas layer because both the oil and the therapeutic agent are hydrophobic. In some embodiments the ratio of drug to oil is 1:2. In some embodiments the ratio of drug to oil is 1:1.

In some embodiments, one or more therapeutic agents are attached to the surface of a microbubble, incorporated in the lipid layer, or trapped within the lipid shell. Microbubbles may further include a targeting agent (e.g., fibrin-targeting agent) or agents that recruit the microbubble to a target site (e.g. site of fibrin deposition in tissue such as an adhesion or plaque). In some embodiments, to rupture the microbubbles and release the therapeutic agent(s), the microbubbles may be irradiated with an energy beam, preferably ultrasonic. If drug release is desired, frequencies used to accomplish drug delivery may range from 0.02 to 40 MHz. Suitable application of ultrasound will result in micro bubble rupture and release of contents. The mechanism of rupture may include but not be limited to large amplitude expansion owing to non-resonant or resonant volume oscillations, inertial collapse, or non-resonant or resonant shape oscillations. Rupture efficiency can also be enhanced by increasing the duration or the strength of the ultrasonic beam.

Either fixed frequency or modulated frequency ultrasound may be used. Fixed frequency is defined wherein the frequency of the sound wave is constant over time. A modulated frequency is one in which the wave frequency changes over time, for example, from high to low (PRICH) or from low to high (CHIRP). Higher peak pressures, such as achieved using tightly focused transducers, may be desired to provide more rapid local delivery of therapeutics. If the microbubbles are produced by microfluidic means as described as one embodiment of this invention, the homogenous nature of the microbubble population will allow efficient bubble rupture and imaging within a narrow frequency range, e.g., 30-40 MHz for Intravascular ultrasound (IVUS), 7-12 MHz for surface vascular ultrasound and 1-3 MHz for clinical echocardiography.

Therapeutic agents may optionally be freed from the microbubbles without rupturing the microbubble. Modest bubble oscillations can allow agents enclosed within the microbubble to pass through the lipid membrane. Agents attached to the lipids may be cleaved from the microbubble surface through enzymatic hydrolysis.

In some embodiments, the microbubbles of the invention retain the therapeutic agent under physiological conditions. In some embodiments, the microbubbles of the invention retain 50%, 55%, 60%, 70%, 80%, 90%, 95%, 99% of the therapeutic agent. In some embodiments, the microbubbles of the invention retain at least 70% of the therapeutic agent. In some embodiments, the microbubbles of the invention retain 80% of the therapeutic agent. In some embodiments, the microbubbles of the invention retain 90% of the therapeutic agent. In some embodiments, the microbubbles of the invention retain 100% of the therapeutic agent.

In some embodiments, without intending to be limited to any theory, polymerization prevents the therapeutic agent leakage for days under physiological conditions. The partially or completely polymerized microbubbles of the invention are stable against leakage yet capable of instantaneous release for remote controlled drug delivery. Polymerization increases not only the stability in solution but also the stability under ultrasound (dissolution rate), offering greater mechanical stability to help counter microbubble destruction. The dissolution rate is tunable by controlling the amount of polymer in the shell.

In some embodiments, the invention provides microbubbles to be imaged at one ultrasound frequency and release their payload at another. In some embodiments, microbubble shell properties are optimized to maximize efficiency for a given application, increasing or decreasing stiffness to maximize binding at a target site or modulating stability to optimize delivery.

### Linking Carriers

In some embodiments, the microbubbles comprise a linking carrier. The term linking carrier includes entities that serve to link agents, e.g., targeting agents and/or therapeutic agents, to the microbubbles. In some embodiments, the linking carrier serves to link a therapeutic agent and the targeting agent. In some embodiments, the linking carrier confers additional advantageous properties to the microbubbles. Examples of these additional advantages include, but are not limited to: 1) multivalency, which is defined as the ability to attach either i) multiple therapeutic agents and/or targeting agents to the microbubbles (e.g., several units of the same therapeutic agent, or one or more units of different therapeutic entities), which increases the effective “payload” of the therapeutic entity delivered to the targeted site; ii) multiple targeting agents to microbubble (e.g., one or more units of the same or different therapeutic agents); and 2) improved circulation lifetimes, which can include tuning the size of the particle to achieve a specific rate of clearance by the reticuloendothelial system.

In some embodiments, the linking carriers are biocompatible polymers (such as dextran) or macromolecular assemblies of biocompatible components (such as microbubbles). Examples of linking carriers include, but are not limited to, microbubbles, polymerized microbubbles, other lipid vesicles, dendrimers, polyethylene glycol assemblies, capped polylysines, poly(hydroxybutyric acid), dextrans, and coated polymers. A preferred linking carrier is a polymerized microbubble. Another preferred linking carrier is a dendrimer.

The linking carrier can be coupled to the targeting agent and/or the therapeutic agent by a variety of methods, depending on the specific chemistry involved. The coupling can be covalent or non-covalent.

A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

### Stabilizing Entities

In some embodiments, the microbubbles contain a stabilizing entity. As used herein, “stabilizing” refers to the ability to impart additional advantages to the microbubbles, for example, physical stability, i.e., longer half-life, colloidal stability, and/or capacity for multivalency; that is, increased payload capacity due to numerous sites for attachment of targeting agents. Stabilizing entities include macromolecules or polymers, which may optionally contain chemical functionality for the association of the stabilizing entity to the surface of the microbubble, and/or for subsequent association of therapeutic agents and/or targeting agents. The polymer should be biocompatible with aqueous solutions. Polymers useful to stabilize the microbubbles of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolyner or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include proteins, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polydiacetylene, polyethylenes (such as, for example, polyethylene glycol, the class of compounds referred to as Pluronics, commercially available from BASF, (Parsippany, N.J.), polyoxyethylene, and polyethylene terephthalate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of microbubbles which employ polymers to stabilize microbubble compositions will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

In some embodiments, the stabilizing entity is dextran. In some embodiments, the stabilizing entity is a modified dextran, such as amino dextran. In a further preferred embodiment, the stabilizing entity is poly(ethylene imine) (PEI). Without being bound by theory, it is believed that dextran may increase circulation times of microbubbles in a manner similar to PEG. Additionally, each polymer chain (i.e. aminodextran or succinylated aminodextran) contains numerous sites for attachment of targeting agents, providing the ability to increase the payload of the entire lipid construct. This ability to increase the payload differentiates the stabilizing agents of the present invention from PEG. In most instances of PEG there is only one site of attachment, thus the targeting agent loading capacity for PEG (with a single site for attachment per chain) is limited relative to a polymer system with multiple sites for attachment. However, branched PEGs are also available and occasionally used. That will increase the number of targeting agents per PEG chain.

In some embodiments, the following polymers and their derivatives are used poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), polyvinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the microbubble or other molecule.

In some embodiments, the polymer may act as a hetero- or homobifunctional linking agent for the attachment of targeting agents, therapeutic entities, proteins or chelators such as DTPA and its derivatives.

In some embodiments, the stabilizing entity is associated with the microbubble by covalent means. In another embodiment, the stabilizing entity is associated with the microbubble by non-covalent means. Covalent means for attaching the targeting entity with the microbubbles are known in the art and described in the US publication 2010/0111840 entitled Stabilized Therapeutic and Imaging Agents, incorporated by reference herein in its entirety.

Noncovalent means for attaching the targeting entity with the microbubble include but are not limited to attachment via ionic, hydrogen-bonding interactions, including those mediated by water molecules or other solvents, hydrophobic interactions, or any combination of these.

In some embodiments, the stabilizing agent forms a coating on the microbubble.

In some embodiments, the microbubbles of the invention may also be linked to functional agents, such as poly(ethylene glycol) (PEG), that otherwise modify microbubble properties, such as aggregation tendencies, binding by opsonizing plasma proteins, uptake by cells, and stability in the bloodstream.

### Methods for Producing Microbubbles

The microbubbles described herein may be prepared in any suitable manner known to practitioners of the art, such as by sonication, vacuum drying, shaking of a lipid solution in the presence of a gas. In some embodiments, microbubbles described herein are prepared through microfluidic flow focusing of a gas into an aqueous solution of the encompassing lipids. If the microbubbles produced form a population heterogenous in size, the size of the microbubbles may be further adjusted, such as by extrusion through a filter with a fixed pore size. Centrifugation of a polydisperse collection of microbubbles may also be used to separate microbubbles by size.

Upon assembly, microbubbles may be polymerized by UV light, e.g., for 2-5 minutes, or any other means for polymerization, depending on the crosslinking moiety on the polymerizable lipid(s). In some embodiments, microbubbles may be polymerized by UV light for 2, 5, 10, 15, 20, 30, 45, 50 or 60 minutes. In some embodiments, microbubbles may be polymerized by UV light for 1, 2, 5, 10, or 15 hours. The longer the UV exposure the more rigid the microbubble will be. The length of the UV exposure will vary depending on the composition and the application of the microbubbles. The UV wavelength can be in the range of UV wavelength: 200-400 nm.

In some embodiments, the microbubbles described herein are produced by microfluidic flow focusing. Microfluidic flow focusing is a method for generating emulsions by flowing immiscible fluids through a small aperture, causing a pinching off of particles at regular intervals due to physical constraints. This method has been used successfully to generate microemulsions (Anna et al. 2003, Appl. Phys. Lett. 82, 364-366; Gafian-Calvo et al. 2001, Phys. Rev. Lett. 87, 274501; Garstecki et al. 2005, Phys. Rev. Lett. 94, 164501; Cubaud et al. 2005, Phys. Rev. E 72, 037302). It has been shown that viscosity controls size and distribution of particles (De Menech et al. 2008, J. Fluid Mech. 595, 141-161). Thus, by varying the viscosity of the lipid solution, microbubble size and size distribution can be varied. For water in oil emulsions, flow rate and ratio of flows have been shown to control the size of particles (Anna et al. 2003, Appl. Phys. Lett. 82, 364-366).

In some embodiments, the gas phase is decafluorobutane, and the liquid phase is lipids in aqueous solution. The gas phase, decafluorobutane is forced through the aperture, creating gas-filled microbubbles. This method generates microbubbles with size distributions subject to control through various parameters.

In some embodiments, a microfluidic flow focusing is applied in order to generate polymerized shell microbubbles for use as ultrasound contrast agents. Current techniques use sonication to generate particles, resulting in large polydispersity due to lack of control. Using flow focusing, narrow distributions of particles can be generated, and size can be controlled through various parameters to generate particles of ideal size. In an exemplary embodiment, a single emulsion microfluidic device using AUTOCAD is used as described in U.S. Patent Application Publication No. 2013/0129635, herein incorporated by reference. Monodisperse microbubbles can also be produced using multichannel microfluidic chips with step-emulsification (see, e.g., Li et al. (2015) Lab Chip. 2015 Feb. 21; 15(4):1023-31; herein incorporated by reference).

In some embodiments, the microbubbles comprise one or more polymerizable lipids. The stability of polymerized shell lipid microbubbles can be increased by increasing the mole fraction of polymerized lipids in the lipid shell. Examples of polymerizable lipids include but are not limited to, diyne PC and diynePE, for example, 1,2-bis(10,12-tricosadiynoyl-sn-glycero-3-phosphocoline. In one embodiment, the polymerized lipid shell of the microbubble comprises at least one polymerizable lipid and at least one non-polymerizable lipid and has a percentage of about 5-50% polymerizable lipid. In some embodiments, the percentage of polymerizable lipid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% of the total lipid mixture making up the microbubbles. In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of polymerizable lipids. In some embodiments, the microbubbles of the invention comprise at least 25% polymerizable lipids. In some embodiments, the microbubbles of the invention comprise at least 50% polymerizable lipids. In one embodiment, at least one polymerizable lipid is a diacetylenic lipid.

In some embodiments, the microbubbles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG).

In another embodiment, the at least one non-polymerizable lipid is selected group the group of L-α-phosphatidylcholine, PE-PEG2000 (1,2-distearoyl-sn-glycero-3-phosphoethanolamine-N-[methoxy(polyethylene glycol)-2000 or PE-PEG2000-biotin. In one embodiment, the polymerized lipid shell comprises a percentage of PEGylated lipid between about 1-20%. In some embodiments, the percentage of PEGylated lipid in the microbubble is 1, 2, 3, 4, 5, 6, 7, 8, 9, 10, 11, 12, 13, 14, 15, 16, 17, 18, 19, or 20. In one embodiment, the lipid is non-polymerizable and PEGylated. In another embodiment, the lipid is polymerizable and PEGylated.

In some embodiments, the microbubbles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the microbubbles of the invention comprise at least 2% of negatively charged lipids. In some embodiments, the microbubbles of the invention comprise at least 5% negatively charged lipids. In some embodiments, the microbubbles of the invention comprise at least 10% negatively charged lipids. In some embodiments, the microbubbles of the invention comprise at least 25% negatively charged lipids. In some embodiments, the microbubbles of the invention comprise at least 30% negatively charged lipids.

In some embodiments, the microbubbles of the invention comprise at least 2% negatively charged lipids and at least 5-50% of a polymerizable lipid. In some embodiments, the microbubbles of the invention comprise at least 5% of negatively charged lipids and at least 5-50% of a polymerizable lipid. In some embodiments, the microbubbles of the invention comprise at least 10% of negatively charged lipids and at least 5-50% of a polymerizable lipid. In some embodiments, the microbubbles of the invention comprise at least 25% of negatively charged lipids and at least 5-50% of a polymerizable lipid. In some embodiments, the microbubbles of the invention comprise at least 30% of negatively charged lipids and at least 5-50% of a polymerizable lipid. In some embodiments, the microbubbles of the invention comprise the same percentage of negatively charged lipids and polymerizable lipids. In some embodiments, the negatively charged lipid and the polymerizable lipid is the same. In some embodiments, the microbubbles of the invention comprise at least two negatively charged lipids, but only one of the two negatively charged lipids is polymerizable.

In one embodiment, the gas of the microbubble is a heavy gas. In one embodiment, the heavy gas is a perfluorocarbon. In another embodiment, the gas of the microbubble is a mixture of at least two perfluorocarbons. Perfluorocarbons (PFCs) are fluorocarbons, compounds derived from hydrocarbons by replacement of hydrogen atoms by fluorine atoms. PFCs are made up of carbon and fluorine atoms only, such as octafluoropropane, perfluorohexane and perfluorodecalin. A perfluorocarbon can be arranged in a linear, cyclic, or polycyclic shape. Perfluorocarbon derivatives are perfluorocarbons with some functional group attached, for example perfluorooctanesulfonic acid. Perfluorocarbon derivatives can be very different from perfluorocarbons in their properties, applications and toxicity. Other examples of perfluorocarbons are tetrafluoromethane, hexafluoroethane, octafluoropropane (perfluoropropane), perfluorocyclobutane, perfluoro-n-butane, and perfluoro-iso-butane. In some embodiments, the perfluorocarbon is decafluorobutane.

In some embodiments, the microbubble has a diameter size range that is about 1 μm-20 μm. In some embodiments, the microbubble has a diameter size range that is about 1 μm-10 μm. In some embodiments, the microbubble has a diameter size range that is about 0.1 μm-150 μm. In some embodiments, the microbubble has a diameter size range that is about 3-5 μm. In some embodiments, the microbubble has a diameter size range of about 2 μm. In some embodiments, the microbubble has a diameter size of about 3 μm. In another embodiment, the microbubble has a diameter size of about 4 μm. In one embodiment, the microbubble has a diameter size of about 3-5 μm. In other embodiments, the microbubble has a diameter size of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 μm.

In one embodiment, the microbubble has an absorbance at a wavelength between about 400-580 μm. In other embodiments, the microbubble has an absorbance at a wavelength between about 400-650 μm. In some embodiments, the microbubble has an absorbance at a wavelength of about 400, 410, 420, 430, 440, 450, 460, 470, 480, 490, 500, 510, 520, 530, 540, 550, 560, 570, 580, or 650 μm. In one embodiment, the absorbance at a wavelength between about 400-580 nm is an indication of the successful polymerization of the polymerizable lipid forming the shell of the microbubble. In other embodiments, the absorbance at a wavelength between about 400-650 nm is an indication of the successful polymerization of the polymerizable lipid forming the shell of the microbubble. This is especially so when the polymerizable lipid is a diacetylenic lipid. In another embodiment, the microbubble appears to be blue or purple, wherein blue indicates one form of polymerized diacetylenic lipid and purple indicates a mixture of a red and a blue form of polymerized diacetylenic lipid.

In one embodiment, the microbubble is UV treated for about 2-5 minutes after fabrication to polymerize the lipid shell. It is understood that one can UV treat the formed microbubbles for a time period of anywhere from 2.0 minutes to several hours in order to achieve various/desired level of polymerization in the shell. In some embodiments, the microbubble is UV treated for about 2, 5, 10, 15, 20, 30, 45, 50 or 60 minutes. In some embodiments, the microbubble is UV treated for about 1, 2, 5, 10, or 15 hours. The UV wavelength can be in the range of UV wavelength: 200-400 nm. The shell material affects microbubble mechanical elasticity. The more elastic the material, the more acoustic energy it can withstand before bursting (McCulloch et al., 2000, J Am Soc Echocardiogr. 13: 959-67).

The level of polymerization of the shell affects the mechanical elasticity. By varying the UV treatment timing, the amount of polymerization of the shell can be adjusted, e.g. 2 or 3 minutes for lower polymerization, 4-30 minutes for higher polymerization. In one embodiment, the microbubble is UV treated for about 2 minutes. In another embodiment, the microbubble is UV treated for about 3 minutes. In another embodiment, the microbubble is UV treated for about 4 minutes. In another embodiment, the microbubble is UV treated for about 5 minutes. In other embodiments, the microbubble is UV treated for about 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 minutes. In another embodiment, the microbubble is UV treated for about 10 minutes. In another embodiment, the microbubble is UV treated for about 20 minutes. In another embodiment, the microbubble is UV treated for about 30 minutes. In another embodiment, the microbubble is UV treated for about 60 minutes. In another embodiment, the microbubble is UV treated for about 2 hours.

In some embodiments, the microbubbles of the invention remain intact after 2, 3, 4, 5, 7, 8, 9 10, 20, 30, 40, 50, 60, 70, 90 minutes. In some embodiments, the microbubbles of the invention remain intact after 2, 3, 4, 5, 7, 8, 9 10, 15, 20, 30, 40, 50, 60, 70, 90 hours. In some embodiments, the microbubbles of the invention remain intact after two days.

In some embodiments, the microbubbles of the invention are used as ultrasound contrast agents. In some embodiments, the microbubbles retain at least 30%, 35%, 40%, 45%, 50%, 60%, 70%, 75%, 80%, or 90% of their signal after two seconds of ultrasound insonation. In some embodiments, the microbubbles retain at least 90% of their signal after two seconds of ultrasound insonation. In some embodiments, the microbubbles retain at least 30%, 35%, 40%, 45%, 50%, 60%, 70%, 75%, 80%, or 90% of their signal after 1, 2, 3, 4, 5, 6, 7, 8, 9, 10, 15 minutes of ultrasound insonation. In some embodiments, the microbubbles retain at least 75% of their signal after 10 minutes of ultrasound insonation. In some embodiments, the microbubbles retain at least 75% of their signal after 15 minutes of ultrasound insonation. In some embodiments, the microbubbles retain at least 90% of their signal after 10 minutes of ultrasound insonation. In some embodiments, the microbubbles retain at least 90% of their signal after 15 minutes of ultrasound insonation. In some embodiments, the microbubbles retain at least 75% of their signal after 1 hour or more of ultrasound insonation. In some embodiments, the microbubbles retain at least 75% of their signal after 2 hours or more of ultrasound insonation. In some embodiments, the microbubbles retain at least 90% of their signal after 1 hour or more of ultrasound insonation. In some embodiments, the microbubbles retain at least 90% of their signal after 2 hours or more of ultrasound insonation. In some embodiments, the microbubbles retain at least 90% of their signal after 3 or 4 hours. In some embodiments, the microbubbles are selectively destroyed at frequencies other than the imaging frequency.

In some embodiments, the microbubbles of the invention have increased circulation time. In some embodiments, the microbubbles of the invention remain intact in circulation after 2, 3, 4, 5, 7, 8, 9 10, 20, 30, 40, 50, 60, 70, 90 minutes. In some embodiments, the microbubbles of the invention remain intact in circulation after 2, 3, 4, 5, 7, 8, 9 10, 15, 20, 30, 40, 50, 60, 70, 90 hours. In some embodiments, the microbubbles of the invention remain intact in circulation after two days. In some embodiments, the microbubbles of the invention are cleared from the system after the targeted microbubbles have been destroyed, e.g., by ultrasound insonication after 3 or 4 hours of administration.

In some embodiments, the microbubbles of the invention have increased half-life. In some embodiments, the microbubbles of the invention have a half-life of 2, 3, 4, 5, 7, 8, 9 10, 20, 30, 40, 50, 60, 70, 90 minutes. In some embodiments, the microbubbles of the invention have a half-life of 2, 3, 4, 5, 7, 8, 9 10, 15, 20, 30, 40, 50, 60, 70, 90 hours. In some embodiments, the microbubbles of the invention have a half-life of two days. In some embodiments, the microbubbles of the invention have a half-life of two hours. In some embodiments, 90% of the microbubbles remain intact after 1 hour but are completely cleared from the system after 3 to 4 hours after administration. In some embodiments, the microbubbles are completely cleared from the system not before 1 hour but are cleared after 3 to 4 hours after administration.

### Disparity

In some embodiments, the invention provides methods to improve signal by optimizing the fibrin-targeted ultrasound contrast agents (USCA) through control of average size, polydispersity, and stability in the bloodstream, thereby increasing their potential applicability. Current techniques for producing microbubble contrast agents that involve sonication or agitation result in large polydispersity and batch-to-batch variation. Monodisperse microbubbles are desirable because they result in a more uniform acoustic response, greater echogenicity, and in the case of a contrast agent carrying a payload, a more selective drug release profile. In some embodiments, monodisperse microbubbles are produced by microfluidic flow focusing. Microfluidic flow focusing has the potential to create particles of narrow size distributions, which can be controlled by adjusting the flow rates of the two impinging fluids.

In some embodiments, by using monodisperse PSMs the resolution can be increased while tuning the shell rigidity to optimize microbubble properties for a given application. In addition to the proximal imaging applications, the technology can be used for drug delivery and gene delivery. By tuning the shell rigidity, and thus ultrasound stability, the PSMs might be engineered to be visualized at one ultrasound frequency and destroyed at a different frequency. Microbubble stability in ultrasound has been shown to directly affect gene transfer efficiency (Alter et al. 2009, Ultrasound Med. Biol. 35, 976-984).

In some embodiments, the microbubble shell composition improves stability in the bloodstream, thereby increasing blood circulation time. In some embodiments, the composition also affects ultrasound echogenicity by changing the surface elasticity, surface tension, or microstructure. Thus, without intending to be limited to any theory, the invention provides an enhanced acoustic response in the region of interest by using an optimal microbubble shell for targeted USCA.

In another embodiment, the present invention provides for a collection of microbubbles comprising gas-filled polymerized shell lipid microbubbles, wherein the microbubbles in the collection are monodispersed and are within a micrometer size range. In one embodiment, the microbubbles of the collection have all the characteristics of a microbubble described herein. In some embodiments, the collection of monodispersed microbubbles is generated by a microfluidic flow focusing method.

In one embodiment, the collection of microbubbles is monodispersed, and the monodisperity is about 20% of an average size of the microbubbles in the collection. In one embodiment, the collection of microbubbles is monodispersed, and the monodisperity is about 15% of an average size of the microbubbles in the collection. In one embodiment, the collection of microbubbles is monodispersed, and the monodisperity is about 10% of an average size of the microbubbles in the collection. In one embodiment, the collection of microbubbles is monodispersed, and the monodisperity is about 5% of an average size of the microbubbles in the collection. In one embodiment, the collection of microbubbles is monodispersed, and the monodisperity is about 1% of an average size of the microbubbles in the collection.

In some embodiments, 90% of the microbubbles in the collection remain intact after 2, 3, 4, 5, 7, 8, 9 10, 20, 30, 40, 50, 60, 70, 90 minutes. In some embodiments, 90% of the microbubbles in the collection remain intact after 2, 3, 4, 5, 7, 8, 9 10, 15, 20, 30, 40, 50, 60, 70, 90 hours. In some embodiments, 90% of the microbubbles in the collection remain intact after two days. In some embodiments, 80% of the microbubbles in the collection remain intact after 2, 3, 4, 5, 7, 8, 9 10, 20, 30, 40, 50, 60, 70, 90 minutes. In some embodiments, 80% of the microbubbles in the collection remain intact after 2, 3, 4, 5, 7, 8, 9 10, 15, 20, 30, 40, 50, 60, 70, 90 hours. In some embodiments, 80% of the microbubbles in the collection remain intact after two days. In some embodiments, 50% of the microbubbles in the collection remain intact after 2, 3, 4, 5, 7, 8, 9 10, 20, 30, 40, 50, 60, 70, 90 minutes. In some embodiments, 50% of the microbubbles in the collection remain intact after 2, 3, 4, 5, 7, 8, 9 10, 15, 20, 30, 40, 50, 60, 70, 90 hours. In some embodiments, 50% of the microbubbles in the collection remain intact after two days. In some embodiments, 90% of the microbubbles in the collection remain intact after 90 minutes. In some embodiments, 50% of the microbubbles in the collection remain intact after 15 hours. In some embodiments, 50% of the microbubbles in the collection remain intact after two days.

In some embodiments, the collection of microbubbles of the invention is used as ultrasound contrast agents. In some embodiments, the collection of microbubbles retains at least 30%, 35%, 40%, 45%, 50%, 60%, 70%, 75%, 80%, or 90% of its signal after two seconds of ultrasound insonation. In some embodiments, the collection of microbubbles retains at least 90% of its signal after two seconds of ultrasound insonation. In some embodiments, the collection of microbubbles retains at least 30%, 35%, 40%, 45%, 50%, 60%, 70%, 75%, 80%, or 90% of its signal after 1, 2, 3, 4, 5, 6, 7, 8, 9, 10, 15 minutes of ultrasound insonation. In some embodiments, the collection of microbubbles retains at least 75% of its signal after 10 minutes of ultrasound insonation. In some embodiments, the collection of microbubbles retains at least 75% of its signal after 15 minutes of ultrasound insonation. In some embodiments, the collection of microbubbles retains at least 90% of its signal after 10 minutes of ultrasound insonation. In some embodiments, the collection of microbubbles retains at least 90% of its signal after 15 minutes of ultrasound insonation. In some embodiments, the collection of microbubbles retains at least 75% of its signal after 1 hour or more of ultrasound insonation. In some embodiments, the collection of microbubbles retains at least 75% of its signal after 2 hours or more of ultrasound insonation. In some embodiments, the collection of microbubbles retains at least 90% of its signal after 1 hour or more of ultrasound insonation. In some embodiments, the collection of microbubbles retains at least 90% of its signal after 2 hours or more of ultrasound insonation. In some embodiments, the collection of microbubbles retains at least 90% of its signal for at least 30 minutes for the diagnostic imaging session and then the desired targeted microbubbles are destroyed. The rest of the microbubbles are cleared from the system about 3 to 4 hours later without them being destroyed.

In some embodiments, the collection of microbubbles of the invention has increased circulation time. In some embodiments, the collection of microbubbles of the invention remains intact in circulation after 2, 3, 4, 5, 7, 8, 9 10, 20, 30, 40, 50, 60, 70, 90 minutes. In some embodiments, the collection of microbubbles of the invention remains intact in circulation after 2, 3, 4, 5, 7, 8, 9 10, 15, 20, 30, 40, 50, 60, 70, 90 hours. In some embodiments, the collection of microbubbles of the invention remains intact in circulation after two days. In some embodiments, 90% of the collection of microbubbles of the invention remains intact in circulation after about 3 to 4 hours.

In some embodiments, the collection of microbubbles of the invention has increased half-life. In some embodiments, the collection of microbubbles of the invention has a half-life of 2, 3, 4, 5, 7, 8, 9 10, 20, 30, 40, 50, 60, 70, 90 minutes. In some embodiments, the collection of microbubbles of the invention has a half-life of 2, 3, 4, 5, 7, 8, 9 10, 15, 20, 30, 40, 50, 60, 70, 90 hours. In some embodiments, the collection of microbubbles of the invention has a half-life of two days. In some embodiments, of the collection of microbubbles of the invention remains intact in circulation after about 3 to 4 hours, but then are cleared from the system.

In some embodiments, the microbubble has a diameter size range that is about 1 μm-20 μm. In some embodiments, the microbubble has a diameter size range that is about 1 μm-10 μm. In some embodiments, the average size of the microbubbles in the collection is between about 0.1 μm-150 μm. In some embodiments, the average size of the microbubbles in the collection is between about 3-5 μm. In some embodiments, the average size of the microbubbles in the collection is about 2 μm. In some embodiments, the average size of the microbubbles in the collection is about 3 minutes.

In another embodiment, the average size of the microbubbles in the collection is about 4 μm. In one embodiment, the average size of the microbubbles in the collection is between about 3-5 μm. In other embodiments, the average size of the microbubbles in the collection is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 μm.

In some embodiments, the microbubbles of the collection comprise at least 25% of polymerizable lipids. In some embodiments, the microbubbles of the collection comprise at least 50% of polymerizable lipids. In one embodiment, the at least one polymerizable lipid is a diacetylenic lipid

In some embodiments, the microbubbles of the collection comprise one or more negatively charged lipids. In one embodiment, the microbubbles of the collection comprise a percentage of PEGylated lipid between about 1-20%.

In some embodiments, the microbubbles of the collection comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the microbubbles of the collection comprise at least 2% of negatively charged lipids. In some embodiments, the microbubbles of the collection comprise at least 5% of negatively charged lipids. In some embodiments, the microbubbles of the collection comprise at least 10% of negatively charged lipids. In some embodiments, the microbubbles of the collection comprise at least 25% of negatively charged lipids. In some embodiments, the microbubbles of the collection comprise at least 30% of negatively charged lipids.

In some embodiments, the microbubbles of the collection comprise at least 2% of negatively charged lipids and at least 5-50% of a polymerizable lipid. In some embodiments, the microbubbles of the collection comprise at least 5% of negatively charged lipids and at least 5-50% of a polymerizable lipid. In some embodiments, the microbubbles of the collection comprise at least 10% of negatively charged lipids and at least 5-50% of a polymerizable lipid. In some embodiments, the microbubbles of the collection comprise at least 25% of negatively charged lipids and at least 5-50% of a polymerizable lipid. In some embodiments, the microbubbles of the collection comprise at least 30% of negatively charged lipids and at least 5-50% of a polymerizable lipid. In some embodiments, the microbubbles of the collection comprise the same percentage of negatively charged lipids and polymerizable lipids. In some embodiments, the negatively charged lipid and the polymerizable lipid is the same.

In some embodiments, the present invention provides for a method of making a microbubble comprising: (a) microfluidic flow focusing a mixture of polymerizable lipid and standard non-polymerizable lipid and a gas through an aperture to form micrometer microbubbles and (b) UV treating the microbubbles of step a to polymerize the polymerizable lipid.

### Compositions

In some embodiments, the invention provides compositions comprising fibrin-targeted microbubbles for the diagnosis and/or treatment of fibrin-associated conditions and disorders. The sizes of the microbubbles may be different for different applications. For general vascular imaging and therapy, sizes may range from about 1 μm to about 20 μm in diameter, preferably between about 2 μm and about 5 μm in diameter. In some embodiments, sizes may range from about 2 μm to about 4 μm. In some embodiments, for applications in tumors or in organs, smaller microbubbles (less than 2 μm in diameter) are preferred. Larger microbubbles may be used for imaging or delivery intrarectally or intranasally, up to about 100 μm in diameter.

In some embodiments, the therapeutic delivery systems of the invention are administered in the form of an aqueous suspension such as in water or a saline solution (e.g., phosphate buffered saline). Preferably, the water is sterile. Also, preferably the saline solution is an isotonic saline solution, although, if desired, the saline solution may be hypotonic (e.g., about 0.3 to about 0.5% NaCl). The solution may also be buffered, if desired, to provide a pH range of about pH 5 to about pH 7.4. In addition, dextrose may be preferably included in the media. Further solutions that may be used for administration of PSMs include, but are not limited to almond oil, corn oil, cottonseed oil, ethyl oleate, isopropyl myristate, isopropyl palmitate, mineral oil, myristyl alcohol, octyldodecanol, olive oil, peanut oil, persic oil, sesame oil, soybean oil, and squalene.

Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

In another embodiment, a controlled release formulation is used that is capable of slowly releasing a composition of the present invention into a subject. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to a subject, form a solid or a gel in situ. Preferred controlled release formulations are biodegradable (i.e., bioerodible).

Generally, the therapeutic/diagnostic agents used in the invention are administered to a subject in an effective amount. Generally, an effective amount is an amount effective to (1) reduce the symptoms of the fibrin-associated condition or disorder sought to be treated, (2) induce a pharmacological change relevant to treating the condition sought to be treated or (3) detect the microbubbles in vivo or in vitro. For an adhesion or plaque, for example, an effective amount includes an amount effective to: reduce the size or break up or eliminate the adhesion or plaque.

Effective amounts of the therapeutic/diagnostic agents can be any amount or dose sufficient to bring about the desired effect and will depend, in part, on the condition, type and location of the adhesion or plaque, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means as described herein, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any subject, preferably to mammals, and more preferably to humans.

The particular mode of administration will depend on the condition to be treated. It is contemplated that administration of the agents of the present invention may be via any bodily fluid, or any target or any tissue accessible through a body fluid.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Polymerized shell microbubbles of this invention may be stored dry or suspended in a variety of liquid solutions, including distilled water or in aqueous solutions. Aqueous solutions may be buffered to suitable pH ranges (about 5 to about 7.4) by HEPES, Tris, phosphate, acetate, citrate, phosphate, bicarbonate, or other buffers, and may contain isotonic (about 0.9% NaCl) or hypotonic (about 0.3 to about 0.5% NaCl) salt concentrations.

The solutions may also include emulsifying and/or solubilizing agents. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propyleneglycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. Suspending and/or viscosity-increasing agents that may be used with lipid or microbubble solutions include but are not limited to, acacia, agar, alginic acid, aluminum monostearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xantham gum.

Bacteriostatic agents may also be included with the microbubbles to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenol, potassium benzoate, potassium sorbate, sodium benzoate and sorbic acid.

### Administration

The methods involve the administration of one or more fibrin-targeted microbubbles, e.g., for the diagnosis and/or treatment of a fibrin-associated condition or disorder. In some embodiments, other agents are also administered, e.g., other therapeutic agents. When two or more agents are co-administered, they may be co-administered in any suitable manner, e.g., as separate compositions, in the same composition, by the same or by different routes of administration.

The compositions of the present invention are typically, although not necessarily, administered via injection (subcutaneously, intravenously, or intramuscularly), by infusion, or locally. Additional modes of administration are also contemplated, such as intra-abdominal, intra-arterial, intravascular, intraperitoneal, intralesional, intracardiac, pericardial, epidural, intralymphatic, interstitial, pulmonary, intratumoral, oral, topical, and so forth. In particular embodiments, compositions are administered locally into the abdomen (e.g., for treatment of an abdominal adhesion), tendon (e.g., for treatment of a peritendinous adhesion), artery (e.g., for treatment of an atherosclerotic plaque) or vein (e.g., for treatment of a venous thrombus) of a subject. The microbubbles may also be utilized in vitro, such as may be useful for diagnosis using tissue biopsies.

In some embodiments, at least one therapeutically effective cycle of treatment with fibrin-targeted microbubbles (e.g., composition comprising polydisperse or monodisperse collection of microbubbles) will be administered to a subject for treatment of a fibrin-associated condition or disorder. By “therapeutically effective cycle of treatment” is intended a cycle of treatment that when administered, brings about a positive therapeutic response with respect to treatment of an individual for a fibrin-associated condition or disorder. Fibrin-targeted microbubbles are administered to a patient in need thereof under conditions wherein the fibrin-targeted microbubbles bind to tissue where fibrin is present, such as at the location of an adhesion or plaque in the patient. Of particular interest is a cycle of treatment wherein the fibrin-targeted microbubbles break up one or more adhesions or plaques upon exposure of the patient to ultrasound in the vicinity of an adhesion or plaque to which the fibrin-targeted microbubbles have bound. In certain embodiments, multiple therapeutically effective doses of compositions comprising fibrin-targeted microbubbles and/or one or more other therapeutic agents or other medications will be administered.

In some embodiments, the microbubbles are administered in a single dose, e.g., for the treatment of an acute condition. Typically, such administration will be by injection. However, other routes may be used as appropriate. In some embodiments, the microbubbles are administered in multiple doses. Dosing may be about once, twice, three times, four times, five times, six times, or more than six times per day. Dosing may be about once a month, once every two weeks, once a week, or once every other day. In one embodiment the microbubbles are administered about once per day to about 6 times per day. In another embodiment, the administration of the microbubbles continues for less than about 7 days. In yet another embodiment, the administration continues for more than about 6, 10, 14, 28 days, two months, six months, or one year. In some cases, continuous dosing is achieved and maintained as long as necessary. In some embodiments, the microbubbles are administered continually or in a pulsatile manner, e.g. with a minipump, patch or stent.

Administration of the microbubbles of the invention may continue as long as necessary. In some embodiments, an agent of the invention is administered for more than 1, 2, 3, 4, 5, 6, 7, 14, 28 days or 1 year. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

When diagnosis and/or treatment need to be performed as a series, e.g., a series of diagnostic tests after treatment, the diagnosis and/or treatment may be performed at fixed intervals, at intervals determined by the status of the most recent diagnostic test or tests or by other characteristics of the individual, or some combination thereof. For example, diagnosis and/or treatment may be performed at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for diagnosis and/or treatment and the availability of diagnostic/treatment facilities, thus approximate intervals corresponding to an intended interval scheme are encompassed by the invention. As an example, an individual who has undergone treatment for a cancer may be tested/treated relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, intervals may be modified.

In one embodiment, a diagnostic test may be performed on an apparently healthy individual during a routine checkup and analyzed so as to provide an assessment of the individual's general health status. In another embodiment, a diagnostic test may be performed to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

Progression in the circulation of the administered microbubble formulation toward the selected site may be monitored any suitable method known in the art, including those described herein, e.g., by ultrasonic imaging means, or by MRI or radiography if the formulation includes agents for such imaging. In some embodiments, the circulation of the administered microbubble formulation toward the selected site is monitored using ultrasonic imaging means. The ultrasonic irradiation may be carried out by a modified echography probe adapted to simultaneously monitor the echo signal and thereby provide an image of the irradiated site. The monitoring signal can be in the range of 100 kHz to 40 MHz and between 2 and 7 MHz.

The useful dosage of gas-filled microbubbles to be administered and the mode of administration will vary depending upon the age, weight, and species of subject to be treated, and the particular application (therapeutic/diagnostic) intended. Typically, dosage is initiated at lower levels and increased until the desired therapeutic effect or imaging visibility is achieved.

### Kits

Any of the compositions described herein may be included in kits. The kits may include the fibrin-targeted microbubbles described herein, or reagents for preparing them, in suitable packaging, and written material that can include instructions for use, discussion of clinical studies, listing of side effects, and the like. Suitable packaging and additional articles for use (e.g., measuring cup for liquid preparations, foil wrapping to minimize exposure to air, and the like) are known in the art and may be included in the kit.

The microbubbles may be provided dry or in a storage solution, and may be pre-polymerized or polymerized before administration, e.g. by UV light exposure. The microbubbles solutions may be ready for administration immediately, or may be suspended or mixed with additional compounds or solutions before administration. The microbubbles provided may already contain therapeutic or contrast agents for usage, or such agents may be linked or incorporated into the microbubbles on-site. Microbubbles may further be provided in specific sizes for different routes of administration or for response to specific ultrasound frequencies, or may be comprised of a heterogeneous distribution of sizes.

The reagents may also include ancillary agents such as buffering agents and stabilizing agents, e.g., polysaccharides and the like. The kit may further include, where necessary, agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of the microbubbles, e.g. ultrasound imaging, which are suitable for the clinical detection, prognosis, and screening of cells and tissue from patients for fibrin-associated conditions and disorders (e.g., adhesions and plaques), as described herein.

Such kits may additionally comprise one or more therapeutic agents. The kit may further comprise a software package for data analysis, which may include reference date for comparison with the test results.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

In one embodiment, the kit comprises fibrin-targeted microbubbles comprising a polymerized lipid shell comprising 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide. In another embodiment, the kit comprises 1,2-distearoyl-sn-glycero-3-phosphoethanolamine-N-[CREKA(polyethylene glycol)-2000] (CREKA-PEG2000-DSPE), 1,2-distearoyl-sn-glycero-3-phosphocholine (DSPC), and 5′-hydroxy-3′-oxypentyl-10-12-pentacosadiynamide for preparing fibrin-targeted microbubbles. In another embodiment, the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25. In another embodiment, fibrin-targeted microbubbles in the kit are monodisperse.

This invention is further illustrated by the following examples which should not be construed as limiting. The contents of all references cited throughout this application, as well as the figures and table are incorporated herein by reference.

### EXAMPLES

### Example 1: Introduction

Targeted contrast ultrasound offers the potential to target specific molecular markers in the body, revealing information about molecular makeup in addition to structure. Information about molecular makeup is crucial in many diagnostic applications, such as surgical adhesions and inflammation in atherosclerosis and angiogenesis in cancer.

The commercially available microbubble preparations currently available are generally designed to have short half-lives (<10 minutes) because they are intended to be used as transient tracers in the bloodstream (Fisher et al. 2002). While sufficiently stable for vascular-based diagnostics, these types of microbubble preparations are likely to be too labile to use for imaging and breaking up adhesions in the peritoneal cavity, where a contrast reagent must be injected in a relatively small volume into the abdomen and requires some time to permeate throughout the peritoneal cavity to find and adhere to its fibrinous-adhesion targets.

In order to solve the stability problem, a microbubble formulation comprising phospholipids and photopolymerizable diacetylene lipids that can be used to produce a microbubble shell matrix that is elastic, non-aggregable, and resistant against gas dissolution was developed. This formulation extends the lifetime of microbubble integrity to hours (rather than minutes).

### Example 2: Preparation of Polymerized Shell Microbubbles

Targeted PSMs were formulated from 1,2-distearoyl-sn-glycero-3-phosphoethanolamine-N-[CREKA(polyethylene glycol)-2000] (“CREKA-PEG2000-DSPE”), 1,2-distearoyl-sn-glycero-3-phosphocholine (DSPC) hydrogenated soy (“soy PC”) and 5′-hydroxy-3′-oxypentyl-10-12-pentacosadiynamide (“h-PEG1-PCDA”) in a molar ratio of 15:60:25. Monodisperse CREKA-PSMs were produced using multichannel microfluidic chips with step-emulsification (Li et al. 2015). The same lipid composition as the polydisperse PSM was used with a nitrogen gas core. Nitrogen gas worked just as well with respect to bubble storage stability and ultrasound response as decafluorobutane, and has the advantage of being quite a bit less expensive. Nitrogen was bubbled through multiple narrow channels (5-7 μm) into a solution of dissolved lipids at pressure (˜15 psi). These microbubbles were then collected and UV cross-linked. The mono-disperse CREKA-PSMs were found to range in size from 1.5-2.5 microns with an average size of 2 microns.

PSMs, formulated in this manner, can be used, for example, in therapeutic applications where cell or tissue sticking or adhesion has a pathological outcome. Areas include breaking up surgically induced adhesions especially in adhesion-caused maladies such as small bowel obstruction, post-surgical morbidity, including chronic abdominal or pelvic pain, infertility in women, and potentially fatal intestinal obstructions. Other therapeutic areas may include dissipation of plaques in atherosclerotic cardiovascular diseases.

### Example 3: Use of Polymerized Shell Microbubbles Diagnostically and Therapeutically for Treatment of Adhesions Resulting from Abdominal Surgery

The current treatment regime for adhesions involves the use of implanted adhesion barrier materials, like Seprafilm (Genzyme, Cambridge, Mass.), a hyaluronate-based material that is applied to adhesion-prone tissue at the time of surgery. When used properly, this technique can reduce the incidence of adhesions by half in certain surgeries (Becker et al., 1996). However, the only current treatment for adhesion-caused maladies such as small bowel obstruction and infertility is adhesiolysis, another invasive surgery that can result in formation of additional adhesions. A non-invasive method of adhesiolysis would be especially exciting as it could break this cycle of adhesion development.

Peritoneal Surgically-Induced Lesion (PSIL) adhesion occurrences can be reduced by performing less invasive surgical procedures (i.e. laparotomy) and using preventative measures such as placing dissolvable film (e.g. Seprafilm) over lesions at the time of surgery. However, the overall incidence of lesion complications remains high even with laparoscopy. Up to 20% of procedures still result in PSIL. Thus, even if it were possible to perform every abdominal procedure by laparotomy, PSIL would still remain a major complication. Furthermore, even if every surgery was performed with correct use of barrier films, about 50% of patients would still develop adhesions. Less invasive surgical techniques and barrier technology cannot completely eliminate adhesion complications, nor do they offer the possibility of diagnostic or post-surgical therapeutic intervention.

From a diagnostic viewpoint, no tests exist that that can specifically locate PSIL, let alone manage it in a timely manner. The consequences of PSIL can sometimes be seen by X-ray without identifying the cause. However, this does not necessarily allow location of the PSIL itself or even that a PSIL may be present or might create a later such complication. Other tests such as computerized tomography (CT) and magnetic resonance imaging (MRI) can provide additional information for more mature or structured abdominal complications, but remain expensive and without direct therapeutic benefit. The secondary use of laparoscopy following such surgery (second look surgery) is an option for some patients but is expensive and still comes with the risk of creating additional adhesions and morbidity. CREKA-PSM offers the potential to utilize ultrasound imaging to diagnose and break up the adhesions that occur in almost 100% of abdominal surgeries, including early-stage adhesions.

### Example 4: Adhesion-Specific Fibrin-Targeted Perfluorobutane Core Microbubbles

The PSMs ability to target fibrin expressed on newly forming fibrinous adhesions in vivo was determined as follows. A pentapeptide with specificity for cells expressing fibrin was identified. This peptide, sequence: CREKA (Zhou 2013), was created with an N-terminal cysteine residue to enable covalent linkage to a maleimide-terminated lipid. The peptide was synthesized and then conjugated to 1,2-distearoyl-sn-glycero-3-phosphoethanolamine-N-[maleimide(polyethylene glycol)-2000] (“mal-PEG2000-DSPE”). The resulting material (“CREKA-PEG-DSPE”) has the CREKA peptide covalently conjugated to the end of a PEG2000 chain, with the distearoyl-phosphoethanolamine lipid connected at the other end of the PEG. This ensures the binding peptide is sterically free to interact with fibrin without interference from the PSM surface.

The CREKA-PEG-DSPE lipids were formulated into PSMs by co-sonication with 1,2-distearoyl-sn-glycero-3-phosphocholine (DSPC) hydrogenated soy (“soy PC”) and 5′-hydroxy-3′-oxypentyl-10-12-pentacosadiynamide (“h-PEG1-PCDA”) in a molar ratio of 15:60:25. The h-PEG₁-PCDA lipid can be photopolymerized by UV light, forming a polydiacetylene polymer. After the initial sonication step in water, the lipid mixture spontaneously formed liposomes. The liposomes were cooled, incubated under pressure with decafluorobutane gas for 48 hours and sonicated to create polydisperse, gas-filled microbubbles. The microbubbles were then irradiated with UV light at 500 mJ/cm² to yield the CREKA-PSM structure, containing the colored polydiacetylene polymer. These CREKA-PSMs were characterized by phase contrast optical microscopy using a Zeiss Axiovert 25 microscope and found to be highly polydisperse with diameters in the 0.5-5 μm range and an average diameter of 1.1 μm.

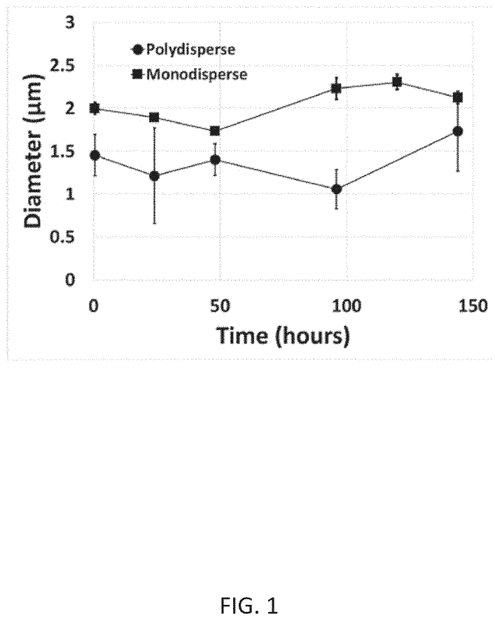

Monodisperse CREKA-PSMs were produced using multichannel microfluidic chips with step-emulsification (Li 2015). The same lipid composition as the polydisperse PSM was used with a nitrogen gas core. Nitrogen gas worked just as well with respect to bubble storage stability and ultrasound response as decafluorobutane, and is quite a bit less expensive. Nitrogen was bubbled through multiple narrow channels (5-7 μm) into a solution of dissolved lipids at pressure (˜15 psi). These microbubbles were then collected and UV cross-linked as described above. Monodisperse CREKA-PSMs were characterized by size and found to be an average of 2 μm, in a range of 1.5-2.5 μm. PSM stability was measured by observing any change in average diameter over the course of 6 days (FIG. 1). Both polydisperse and monodisperse PSMs were found to be exceedingly stable and undergo little size change over this time period.

### Example 5: Evaluating the Stability and Targeting Specificity of Fibrin-Targeted Microbubble Contrast Agents In Vitro

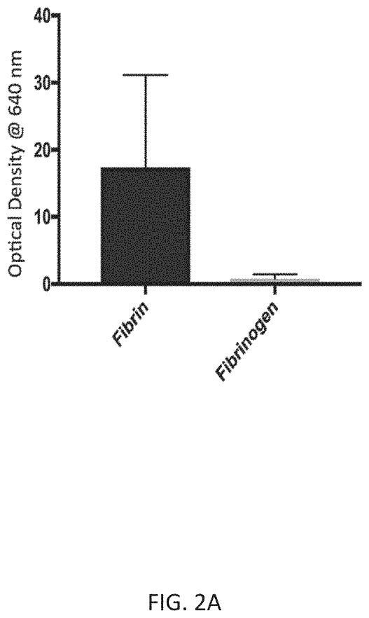

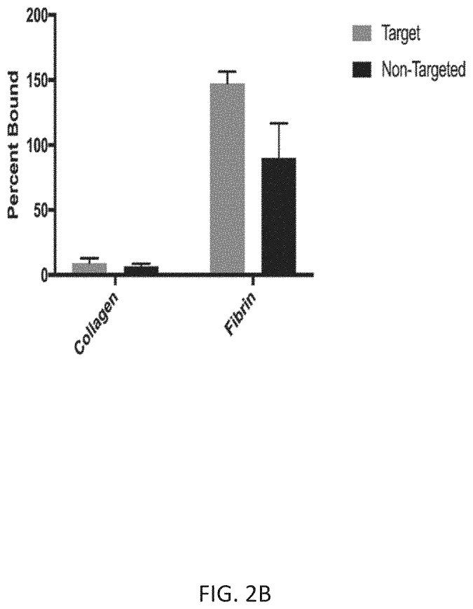

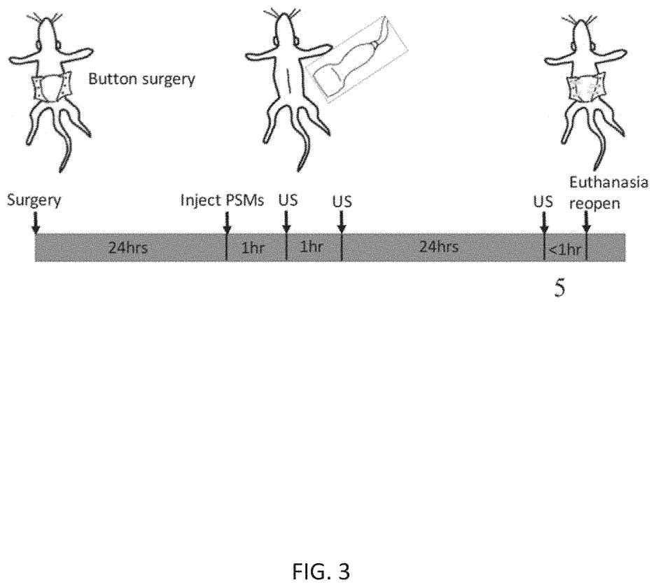

Polydisperse CREKA-PSMs were tested to show specific binding to fibrin in vitro (FIG. 2A) as determined by the change in fluorescence relative to untreated plates. Both targeted and non-targeted PSMs were incubated on fibrin- and fibrinogen-coated 6-well plates and washed to remove unbound PSMs. This experiment was intended to show the binding specificity of CREKA to fibrin versus its precursor molecule fibrinogen. The polydiacetylene polymer (PDA) is inherently fluorescent at about the same wavelength as rhodamine (547 nm absorbance/580 nm emission) and can be used as a handy signal to quantify the amount and presence of the PSMs. As can be seen in FIG. 3A, the CREKA-PSMs bound avidly to fibrin and not to fibrinogen (as expected). This is important as fibrinogen is a common tissue and blood component. Any PSM fibrinogen binding could potentially compete with binding to newly formed adhesions and lead to false positive signals.

Fluorescence of PSM solutions (CREKA-targeted and non-targeted) were measured after exposure to cultured LP-9 mesothelial cells coated with fibrin or a collagen-coated surface (no cells), to determine the binding affinity of PSMs in a biologically active environment (FIG. 2B). Aliquots were removed after PSM incubation, and fluorescence of those samples were measured by fluorimeter readings. The fluorescence signal was compared to the signal of 100% unbound PSMs to determine the percent of PSMs bound. Targeted PSMs showed significantly greater binding to fibrin-coated cells compared to untargeted PSMs. Neither PSM showed any binding to collagen.

### Example 6: In Vivo Imaging of Fibrin-Targeted PSM in a Rat Model of Surgical Adhesions

We performed surgery in 10 male Wistar rats, all approximately 200 g. In 8 of the animals silk sutures were used to create 3 ischemic areas of peritoneal lining on one side of the peritoneum (ischemic buttons). In the remaining two animals, “ablations” were made by scraping and cutting three spots on one side of the peritoneal lining. Both techniques have been shown to lead to abdominal tissue adhesions akin to actual adhesions subsequent to human surgical procedures (Reed 2004; Whang 2011). After surgery, all 10 animals had their abdomen closed and given 24 hours to recover, and the location of the potential adhesion was marked on the untreated side, for 2×10⁵ PSMs total) (FIG. 3).

Included was a negative control animal that never received any surgery. Two animals each were injected with non-targeted polydisperse PSMs, targeted polydisperse PSMs, non-targeted monodisperse PSMs, or targeted monodisperse PSMs. One animal each that received ablation surgery was injected with non-targeted monodisperse PSMs or targeted monodisperse PSMs. The non-operated control animal was injected with targeted monodisperse PSMs. Images were taken on both sides of each animal using a Siemens Accuson Sequoia 512 Ultrasound Machine using a 7V3c transducer at 1, 2, and 24 hours after injection. The ultrasound images taken 24 hours after injection were examined by a trained radiologist who determined whether the pattern of PSMs seen suggested the presence of an adhesion at the site of each button or ablation. Accuracy of these predictions was determined by surgically reopening the animals and observing whether the presence of adhesions correlated with the regions of high observed US signal.

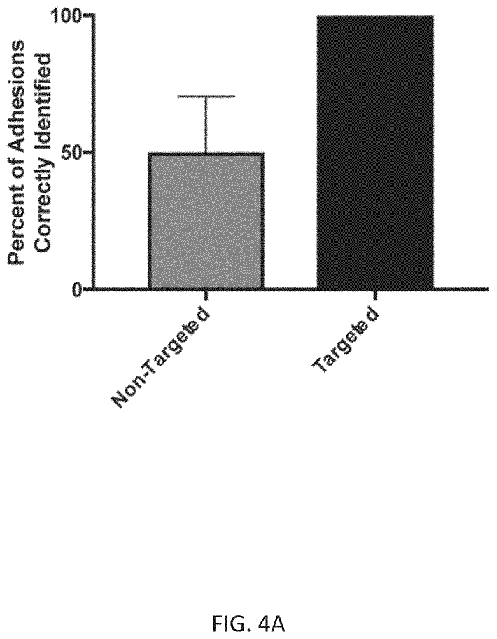

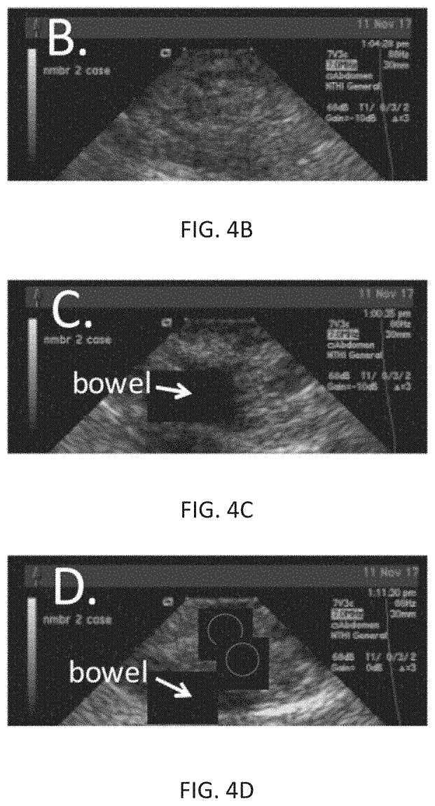

Early ultrasound images (1 and 2 hours after PSM injection) were inconclusive. By 24 hours, distinct PSM localization made it possible to identify regions suggesting the presence of an adhesion. The animal's abdomens were surgically reopened, and the presence of an adhesion was confirmed as well as scored with whether ultrasound measurements detected PSMs localized in the vicinity. With polydisperse PSMs, the following was observed: 1) spots of non-targeted PSMs were generally less consistent and randomly located in injected animals, the scoring was assigned 50% based on positive identification ˜30-50% of the time (statistically random), i.e. half the PSMs were read near the adhesion and half were located in other areas; 2) CREKA-targeted polydisperse PSMs formed clear, consistent bright spots around adhesions by the 24 hour time point (FIG. 4D, circled areas), allowing us to correctly identify 100% of the adhesions in both animals (FIG. 4A). These results demonstrate that polydisperse CREKA-targeted PSMs have the potential to locate and confirm the presence of surgical adhesions in our rat model.

### Example 7: Monodisperse Targeted PSMs Breakup Adhesions Using Ordinary Diagnostic Ultrasound

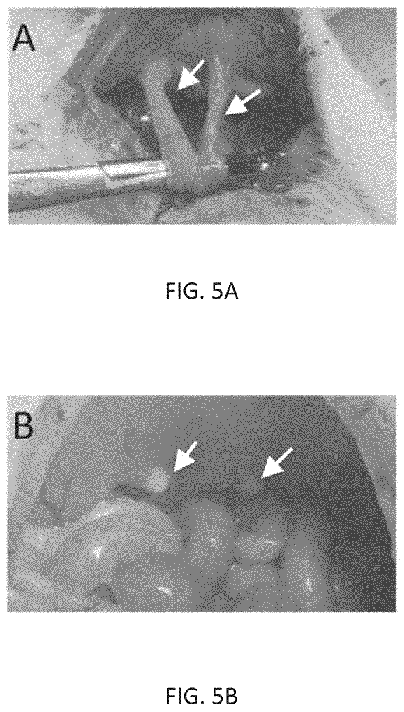

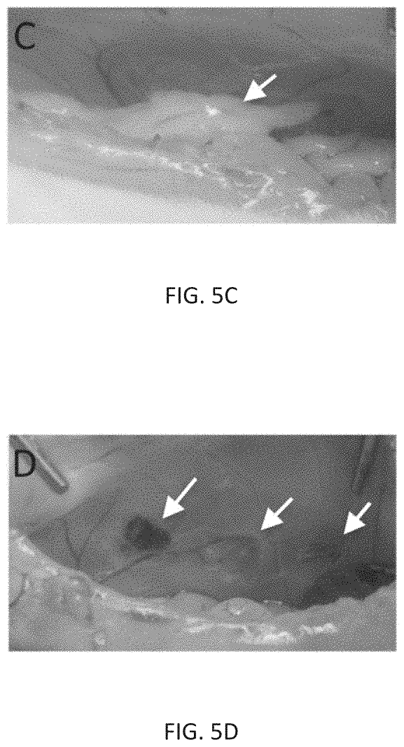

As was done for the animal cohorts that received polydisperse CREKA-PSMs, they again underwent the button or ablation procedure. These animals received monodisperse targeted PSMs and were subjected to the same diagnostic ultrasound. The ultrasound signals taken showed PSM localization marking possible adhesions by the 24-hour time-point. However, no surgical adhesions were found after reopening the animals (FIG. 5), even in a positive control animal subjected to the ablation model. We believe early adhesions did form in these animals, and further hypothesize that US irradiation of monodisperse PSMs in their proximity facilitated their breakup. Adhesion breakup was not seen with non-targeted monodisperse PSMs, further validating the effectiveness of CREKA as a fibrin-targeting molecule in this model. Literature from the field of sonothrombolysis clearly shows that not only therapeutic ultrasound, but also diagnostic ultrasound plus bubbles can breakup clot models after hour-long ultrasound exposures (Zhou 2014).

While the preferred embodiments of the invention have been illustrated and described, it will be appreciated that various changes can be made therein without departing from the spirit and scope of the invention.

1. A fibrin-targeted microbubble comprising:
a) a polymerized lipid shell;
b) a fibrin-targeting agent, wherein the fibrin-targeting agent is conjugated to the polymerized lipid shell; and
c) a gas core comprising at least one gas, wherein the gas core is encased within the polymerized lipid shell.

2. The fibrin-targeted microbubble of claim 1, wherein the polymerized lipid shell comprises at least one polymerizable lipid and at least one non-polymerizable lipid.

3. The fibrin-targeted microbubble of claim 2, wherein the polymerized lipid shell comprises at least about 5% polymerizable lipid.

4. The fibrin-targeted microbubble of claim 3, wherein the polymerizable lipid ranges from about 5 to 50% of the total lipid in the polymerized lipid shell.

5. The fibrin-targeted microbubble of claim 1, wherein at least one non-polymerizable or at least one polymerizable lipid is PEGylated.

6. The fibrin-targeted microbubble of claim 5, wherein PEGylated lipid ranges from about 5% to about 50% of the total lipid in the polymerized lipid shell.

7. The fibrin-targeted microbubble of claim 2, wherein the non-polymerizable lipid is 1,2-distearoyl-sn-glycero-3-phosphocholine (DSPC), 1,2-distearoyl-sn-glycero-3-phosphoethanolamine-N-[methoxy(polyethylene glycol)-2000 (DSPE-PEG2000), or DSPE-PEG2000-biotin.

8. The fibrin-targeted microbubble of claim 2, wherein the polymerizable lipid is a diacetylenic lipid.

9. (canceled)

10. The fibrin-targeted microbubble of claim 1, wherein the fibrin-targeting agent is a peptide, protein, antibody, antibody mimetic, or aptamer that selectively binds to fibrin.

11. The fibrin-targeted microbubble of claim 10, wherein the peptide is a CREKA peptide or CPT-11.

12. The fibrin-targeted microbubble of claim 11, wherein the polymerized lipid shell comprises 1,2-distearoyl-sn-glycero-3-phosphoethanolamine-N-[CREKA(polyethylene glycol)-20001 (CREKA-PEG2000-DSPE), 1,2-distearoyl-sn-glycero-3-phosphocholine (DSPC), and 5′-hydroxy-3′-oxypentyl-10-12-pentacosadiynamide.

13. The fibrin-targeted microbubble of claim 12, wherein the molar ratio of CREKA-PEG2000-DSPE, 1,2-distearoyl-sn-glycero-3-phosphocholine (DSPC), and 5′-hydroxy-3′-oxypentyl-10-12-pentacosadiynamide is 15:60:25.

14. (canceled)

15. (canceled)

16. (canceled)

17. (canceled)

18. (canceled)

19. A composition comprising a collection of microbubbles, wherein the collection comprises the fibrin-targeted microbubble of claim 1.

20. (canceled)

21. (canceled)

22. The composition of claim 19, wherein the microbubbles in the collection are monodisperse or polydisperse microbubbles.

23. (canceled)

24. (canceled)

25. (canceled)

26. (canceled)

27. (canceled)

28. (canceled)

29. A kit comprising the composition of claim 19 and instructions for using the kit for ultrasound imaging for diagnosing or treating a fibrin-associated condition or disorder.

30. (canceled)

31. (canceled)

32. (canceled)

33. (canceled)

34. (canceled)

35. (canceled)

36. A method of treating an adhesion, the method comprising:
a) administering the composition of claim 19 to a patient having an adhesion, under conditions wherein the fibrin-targeted microbubble binds to fibrin on the adhesion in the patient; and
b) exposing the patient to ultrasound in the vicinity of the adhesion to break up the adhesion.

37. The method of claim 36, wherein the adhesion is caused by a surgery, a disease, or a traumatic injury.

38. (canceled)

39. (canceled)

40. The method of claim 36, wherein the fibrin-targeted microbubble further comprises an agent attached to or encapsulated within the polymerized lipid shell.

41. The method of claim 40, wherein the agent is a therapeutic agent or imaging agent.

42. The method of claim 41, wherein the therapeutic agent is a small molecule drug or a therapeutic peptide, protein, or antibody.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)
